SEQUENCE LISTING (38 sequences)

SEQ ID NO: 1 - protein, 37 aa, Hadronyche versuta
SPTCIPSGQPCPYNENCCSQSCTFKENENGNTVKRCD

SEQ ID NO: 2 - protein, 36 aa, Atrax robustus
GACTPTGQPCPYNESCCSGSCQEQLNENGHTVKRCV

SEQ ID NO: 3 - protein, 37 aa, Hadronyche modesta
APVCTRTDQPCPYNEDCCSGSCTLKKNENGNLVKRCD

SEQ ID NO: 4 - protein, 37 aa, Hadronyche modesta
SPVCTRTDQPCPYDQDCCSGSCTLKKNENGNLVKRCD

SEQ ID NO: 5 - protein, 37 aa, Hadronyche modesta
APVCTRTDQPCPYDQDCCSGSCTLKKNENGNLVKRCD

SEQ ID NO: 6 - protein, 36 aa, Hadronyche infensa
STCTPTDQPCPYHESCCSGSCTYKANENGNQVKRCD

SEQ ID NO: 7 - protein, 36 aa, Hadronyche infensa
STCTPTDQPCPYDESCCSGSCTYKANENGNQVKRCD

SEQ ID NO: 8 - protein, 37 aa, Hadronyche infensa
SSTCIRTDQPCPYNESCCSGSCTYKANENGNQVKRCD

SEQ ID NO: 9 - protein, 37 aa, Hadronyche modesta
STVCIPSGQPCPYSKYCCSGSCTYKENENGNTVQRCD

SEQ ID NO: 10 - protein, 37 aa, Hadronyche versuta
SAVCIPSGQPCPYSKYCCSGSCTYKTNENGNSVQRCD

SEQ ID NO: 11 - protein, 37 aa, Hadronyche infensa
SPTCIPTGQPCPYNENCCNQSCTYKANENGNQVKRCD

SEQ ID NO: 12 - protein, 37 aa, Hadronyche infensa
SPTCIPTGQPCPYNENCCSQSCTYKANENGNQVKRCD

SEQ ID NO: 13 - protein, 37 aa, Hadronyche infensa
SPTCIPTGQPCPYNENCCSQSCTYKTNENGNQVKGCD

SEQ ID NO: 14 - protein, 37 aa, Hadronyche infensa
SPTCIPTGQPCPYNENCCSQSCTYKANENGNQVKGCD

SEQ ID NO: 15 - protein, 37 aa, Hadronyche modesta
SPVCIPSGQPCPYNEHCCSGSCTYKENENGNTVQRCD

SEQ ID NO: 16 - protein, 37 aa, Atrax robustus
SSVCIPSGQPCPYNEHCCSGSCTYKENENGNTVQRCD

SEQ ID NO: 17 - protein, 37 aa, Atrax robustus
SPTCIPSGQPCPYNENCCSKSCTYKENENGNTVQRCD

SEQ ID NO: 18 - protein, 37 aa, Hadronyche venenata; identical to SEQ ID NO: 17

SEQ ID NO: 19 - protein, 37 aa, Hadronyche versuta; identical to SEQ ID NO: 17

SEQ ID NO: 20 - protein, 37 aa, Hadronyche formidabilis
SPTCIRSGQPCPYNENCCSQSCTFKTNENGNTVKRCD

SEQ ID NO: 21 - protein, 37 aa, Hadronyche versuta
SSTCIPSGQPCPYNENCCSQSCTYKENENGNTVKRCD

SEQ ID NO: 22 - protein, 37 aa, Hadronyche versuta
SSTCIPSGQPCPYNENCCSQSCTFKENENGNTVKRCD

SEQ ID NO: 23 - protein, 37 aa, Atrax robustus; identical to SEQ ID NO: 1

SEQ ID NO: 24 - protein, 37 aa, Hadronyche versuta
SPTCIPSGQPCPYNENCCSQSCTYKENENGNTVKRCD

SEQ ID NO: 25 - protein, 39 aa, Hadronyche versuta
QYCVPVDQPCSLNTQPCCDDATCTQERNENGHTVYYCRA

SEQ ID NO: 26 - protein, 39 aa, Missulena bradleyi
SPVCTPSGQPCQPNTQPCCNNAEEEQTINCNGNTVYRCA

SEQ ID NO: 27 - protein, 37 aa, Hadronyche versuta
AICPGADRPCAACCPCCPGTSCKAESNGVFYCRKDEP

SEQ ID NO: 28 - protein, 37 aa, Hadronyche versuta
VICTGADSPCAACCPCCPGTSCKAESNGVSYCRKDEP

SEQ ID NO: 29 - protein, 37 aa, Hadronyche versuta
AICTGADRPCAACCPCCPGTSCKAESNGVSYCRKDEP

SEQ ID NO: 30 - protein, 36 aa, Hadronyche versuta
TICTGADRPCAACCPCCPGTSCQGPESNGVVYCRNF

SEQ ID NO: 31 - protein, 37 aa, Hadronyche formidabilis
SPTCTGADRPCAACCPCCPGTSCKGPEPNGVSYCRND

SEQ ID NO: 32 - protein, 36 aa, Hadronyche versuta
TICTGADRPCAACCPCCPGTSCQGPEPNGVSYCRND

SEQ ID NO: 33 - protein, 37 aa, Atrax robustus
SPTCIPSGQPCPYNENCCSQSCTFKENETGNTVKRCD

SEQ ID NO: 34 - protein, 41 aa, Artificial Sequence
  derived from Hadronyche versuta to which amino acids GS are added
GSQYCVPVDQPCSLNTQPCCDDATCTQERNENGHTVYYCRA

SEQ ID NO: 35 - protein, 37 aa, Artificial Sequence
  Mutant 1 derived from w-hexatoxin-Hv1a
SPTCIPSGQPCPYNENCCSQSCTFKENEAGNTVKRCD

SEQ ID NO: 36 - protein, 37 aa, Artificial Sequence
  Mutant 2 derived from w-hexatoxin-Hv1a
SPTCIPSGQPCPYNENCCSQSCTFKENELGNTVKRCD

SEQ ID NO: 37 - protein, 37 aa, Artificial Sequence
  Mutant 3 derived from k-hexatoxin-Hv1c
AICTGADRPCAACCPCCPGTSCKAESLGVSYCRKDEP

SEQ ID NO: 38 - protein, 41 aa, Artificial Sequence
  Mutant 4 derived from GS-w/k-hexatoxin-Hv1h
GSQYCVPVDQPCSLNTQPCCDDATCTQERNELGHTVYYCRA

(19) United States
(12) Patent Application Publication
(10) Pub. No.: US 2024/0076327 A1
(43) Pub. Date: Mar. 7, 2024

(54) PESTICIDAL HEXATOXIN POLYPEPTIDES AND METHODS OF USE THEREOF

(71) Applicant: SYNGENTA CROP PROTECTION AG, Basel (CH)

(72) Inventors: Aurelien BIGOT, Stein (CH); Fides BENFATTI, Stein (CH); David J. CRAIK, St Lucia (AU); Yen-Hua HUANG, St Lucia (AU); Quentin KAAS, St Lucia (AU); Conan WANG, St Lucia (AU)

(73) Assignee: SYNGENTA CROP PROTECTION AG, Basel (CH)

(21) Appl. No.: 18/258,720

(22) PCT Filed: Dec. 21, 2021
(86) PCT No.: PCT/EP2021/087102

§ 371 Date: Jun. 21, 2023

(30) Foreign Application Priority Data
Dec. 21, 2020 (EP) 20216063.6

(51) Int. Cl. C07K 14/435; A01P 7/04

(52) U.S. Cl. CPC C07K 14/43518; A01P 7/04

(57) ABSTRACT

The present invention relates to a hexatoxin peptide variant comprising a. an amino acid sequence which is at least 90% identical to any one of SEQ ID NOs: 27 to 29, wherein the amino acid sequence has at least one amino acid variant on position N27; b. an amino acid sequence which is at least 90% identical to any one of SEQ ID NOs: 2, 6, 7, 30 or 32, wherein the amino acid sequence has at least one amino acid variant on position N28; c. an amino acid sequence which is at least 90% identical to any one of SEQ ID NOs: 1, 3, 4, 5, 8 to 24 or 31, wherein the amino acid sequence has at least one amino acid variant on position N29; d. an amino acid sequence which is at least 90% identical to SEQ ID NO: 25, wherein the amino acid sequence has at least one amino acid variant on position N30; e. an amino acid sequence which is at least 90% identical to SEQ ID NOs: 26, wherein the amino acid sequence has at least one amino acid variant on position N31; or f. an amino acid sequence which is at least 90% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

### FIELD OF THE INVENTION

The present invention describes new variants of spider-derived pesticidal κ-, ω-, κ/ω-hexatoxin and ω-actinopoditoxin peptides, methods for making such peptides, their expression in plants, methods for delivery of the peptides, methods for controlling pests by the peptides, and uses of the peptides for controlling pests. These pesticidal toxin peptide variants have an increased stability at alkaline pH compared to their native peptides, while also being pesticidally active.

### BACKGROUND OF THE INVENTION

Crop loss caused by arthropods and in particular by insects has a major global economic impact. Reduction in yields of wheat, rice, maize, potato and soybean caused by damage associated with 137 pathogens and pests amounts to 17.2%-30% on average (Savary et al., Nature Ecology & Evolution, 2019). This causes a severe strain on the ability to meet the increasing global demand for food production.

Chemical insecticides were introduced during the second half of the 20th century and are to this date still the most important method for reducing damage caused by pests. However, a disadvantage of agrochemicals is considered to be their impact on the environment.

Biopesticides are currently being investigated as safe and effective alternatives to chemical pesticides. These include naturally occurring substances that control pests, such as biochemical pesticides. Insecticidal toxins derived from natural enemies are of particular interest. These include (neuro)toxins produced by spiders, scorpions and sea anemones, (endo)toxins produced by microorganisms such as Bacillus thuringiensis, Xenorhabdus nematophilia and Photorhabdus luminescens, and plant-produced toxins such as defensins.

An issue with these insecticidal toxins is that, when directly applied, there remains a difficulty to reach their target site of interest. The most practical route of application of many of these toxins is through ingestion of the toxin by a target insect. However, the insect's digestive enzymes and the physical barriers of the alimentary canal can prevent uptake of a sufficient amount of insecticidal toxin to reach the target site of interest, and thereby greatly reduces the efficacy of the toxins.

Currently, the most widely applied and most successful bioinsecticides are δ-endotoxins produced by B. thuringiensis. After ingestion by an insect, these toxins bind to the gut endothelium, form cation-selective channels, and cause cell lysis, which then leads to death of the insect. However, a growing concern is the increasing resistance that pest species are developing to these toxins.

Spider venom is a complex composition of enzymes, (neuro)toxins and cytolytic compounds. The majority of insecticidal toxins derived from spiders are disulfide-rich peptide neurotoxins with a size of about 20 to 100 amino acids (Windley et al., Toxins, 2012). The ω-ACTX-Hv1a (also known as ω-hexatoxin-Hv1a) toxin for example has shown an insecticidal effect when expressed in tobacco plants (Khan et al., Transgenic Research, 2006). However, the use of transgenic plants may not be possible or commercially viable in certain jurisdictions. Therefore, alternative means of delivery of insecticidal peptide toxins have to be used. These means can be through common agricultural formulations and application methods such as spraying.

The commercialization of insecticidal peptides for agriculture is difficult because of multiple reasons, one of which is the fact that peptides have a low stability and therefore degrade rapidly. Peptides are preferably stored in lyophilized form at −20° C. or even more preferably at −80° C. or lower temperatures, while peptides in solution are in general only stable for up to a week at 4° C. In addition, a pH of 5 to 7 is considered optimal to preserve stability of peptides in solution. These preferred conditions prohibit the practical storage, formulation and use of insecticidal peptides and compositions comprising these peptides at higher temperatures and/or in an alkaline or highly acidic environment.

To increase the stability of peptides various modifications or compositions have been described in the art. However, these solutions are usually not applicable to all peptides or they relate to an increased form of stability that supplies a marginal advantage (e.g. extreme thermostability). The complex and varied nature of peptides requires a bespoke solution regarding their stability based on their amino acid sequences and three-dimensional structures.

WO2016080367A1 describes a method for improving the stability of an alkaline vaccine composition comprising one or more specific peptides or its variants, a saccharide and an inorganic salt. WO2016080367A1 also discloses that similar stability-increasing compositions might not work on peptides with different amino acid sequences.

U.S. Pat. No. 7,834,158B2 describes an immunoglobulin-binding protein of a certain sequence capable of binding to regions of an immunoglobulin molecule, wherein the protein is mutated by substituting an asparagine for a threonine on position 23, thereby gaining increased stability at alkaline pH-value. However, U.S. Pat. No. 7,834,158B2 also discloses that mutating an asparagine on other positions renders the immunoglobulin-binding protein less suitable to varying degrees.

Hence, there remains a need to identify a means to increase the stability at an alkaline pH-value of pesticidal toxin peptides derived from spiders, while also retaining pesticidal activity.

### SUMMARY OF THE INVENTION

The present invention relates to novel pesticidal, preferably insecticidal, hexatoxin peptides, compositions comprising the peptides, their application to plants, their expression in plants, methods for delivery of the compositions, methods for controlling pest infestations by the compositions, and the use of the compositions for controlling pest infestations.

The peptides, variants, methods and uses of the present invention enhance the stability at alkaline pH of pesticidal κ-, ω- and κ/ω-hexatoxin and ω-actinopoditoxin peptides derived from spiders, while being pesticidally active. As a result they are more useful when stored, easier to formulate or used at alkaline pH and provide an important improvement in the practical application of biopesticides.

In addition, a high luminal pH in the gut has been found in the larvae of a wide range of pests such as lepidopteran, dipteran, termite and beetle (Oryctes nasicornis) larvae (Dow, J. exp. Biol., 1992), which indicates that enhanced stability of pesticidal peptides at an alkaline pH may provide an enhanced pesticidal effect after ingestion of these peptides by the larvae.

In a first aspect, the present invention therefore to provide a hexatoxin peptide variant comprising
a. an amino acid sequence which is at least 90% identical to any one of SEQ ID NOs: 27 to 29, wherein the amino acid sequence has at least one amino acid variant on position N27;
b. an amino acid sequence which is at least 90% identical to any one of SEQ ID NOs: 2, 6, 7, 30 or 32, wherein the amino acid sequence has at least one amino acid variant on position N28;
c. an amino acid sequence which is at least 90% identical to any one of SEQ ID NOs: 1, 3, 4, 5, 8 to 24 or 31, wherein the amino acid sequence has at least one amino acid variant on position N29;
d. an amino acid sequence which is at least 90% identical to SEQ ID NO: 25, wherein the amino acid sequence has at least one amino acid variant on position N30;
e. an amino acid sequence which is at least 90% identical to SEQ ID NOs: 26, wherein the amino acid sequence has at least one amino acid variant on position N31; or
f. an amino acid sequence which is at least 90% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33. These sequences, with exception of SEQ ID NO: 33, are also listed in table 1 herein below.

It is a further object to provide a composition comprising a pesticidally effective amount of a hexatoxin peptide according to the invention and a suitable carrier or diluent.

In another aspect the invention provides a plant, plant tissue or plant propagation material comprising the hexatoxin peptide according to the invention, or having the hexatoxin peptide according to the invention applied thereto.

In a further aspect the invention provides a method for increasing stability of a hexatoxin peptide, the method comprising introducing an amino acid variant on position N27 into an amino acid sequence that is at least 90%, 91%, 92%, 93%, 94%, 95%, 96%, 97%, 98% or 99% identical to an amino acid sequence comprising any one of SEQ ID NOs: 27 to 29, on position N28 into an amino acid sequence that is at least 90%, 91%, 92%, 93%, 94%, 95%, 96%, 97%, 98% or 99% identical to an amino acid sequence comprising any one of SEQ ID NOs: 2, 6, 7, 30 or 32, on position N29 into an amino acid sequence that is at least 90%, 91%, 92%, 93%, 94%, 95%, 96%, 97%, 98% or 99% identical to an amino acid sequence comprising any one of SEQ ID NOs: 1, 3, 4, 5, 8 to 24 or 31, on position N30 into an amino acid sequence that is at least 90%, 91%, 92%, 93%, 94%, 95%, 96%, 97%, 98% or 99% identical to an amino acid sequence comprising SEQ ID NO: 25, on position N31 into an amino acid sequence that is at least 90%, 91%, 92%, 93%, 94%, 95%, 96%, 97%, 98% or 99% identical to an amino acid sequence comprising SEQ ID NOs: 26, or on position N32 into an amino acid sequence that is at least 90%, 91%, 92%, 93%, 94%, 95%, 96%, 97%, 98% or 99% identical to an amino acid sequence comprising SEQ ID NO: 34.

It is a further object to provide a method for delivering a hexatoxin peptide to a pest, the method comprising applying a hexatoxin peptide according to the invention to the plant, plant locus, plant tissue or plant propagation material, to obtain a treated material.

It is yet a further object to provide a method for controlling pest infestations, comprising applying a hexatoxin peptide or a composition according to the invention to a pest or its environment, a plant, plant tissue or plant propagation material or the locus where the plant or plant propagation material is planted.

In a further aspect the invention provides a use of a hexatoxin peptide according to the invention for controlling pest infestations.

### BRIEF DESCRIPTION OF THE SEQUENCE LISTING

SEQ ID NOs: 1 to 34 are sequences derived from or having a spider origin.

SEQ ID NOs: 1, 10, 19, 21, 22, 24, 25, 27 to 30, and 32 are derived from Hadronyche versuta.

SEQ ID NOs: 2, 16, 17, 23 and 33 are derived from Atrax robustus.

SEQ ID NOs: 3, 4, 5, 9 and 15 are derived from Hadronyche modesta.

SEQ ID NOs: 6, 7, 8, 11, 12, 13 and 14 are derived from Hadronyche infensa.

SEQ ID NO: 18 is derived from Hadronyche venenata.

SEQ ID NOs: 20 and 31 are derived from Hadronyche formidabilis.

SEQ ID NO: 26 is derived from Missulena bradleyi.

SEQ ID NO: 34 is derived from Hadronyche versuta to which the amino acids GS are added.

### DETAILED DESCRIPTION OF THE INVENTION

The present invention describes new variants of pesticidal spider-derived κ-, ω- and κ/ω-hexatoxin and ω-actinopoditoxin peptides, methods for making such peptides, their expression in plants, methods for delivery of the peptides, methods for controlling pests by the peptides, and uses of the peptides for controlling pests. These pesticidal hexatoxin peptide variants have an increased stability at alkaline pH compared to their native peptides, while also being pesticidally active. The present invention accordingly relates to a hexatoxin peptide variant comprising
a. an amino acid sequence which is at least 65% identical to any one of SEQ ID NOs: 27 to 29, or homologues thereof, wherein the amino acid sequence has at least one amino acid variant on position N27;
b. an amino acid sequence which is at least 65% identical to any one of SEQ ID NOs: 2, 6, 7, 30 or 32, or homologues thereof, wherein the amino acid sequence has at least one amino acid variant on position N28;
c. an amino acid sequence which is at least 65% identical to any one of SEQ ID NOs: 1, 3, 4, 5, 8 to 24 or 31, or homologues thereof, wherein the amino acid sequence has at least one amino acid variant on position N29;
d. an amino acid sequence which is at least 65% identical to SEQ ID NO: 25, or homologues thereof, wherein the amino acid sequence has at least one amino acid variant on position N30;
e. an amino acid sequence which is at least 65% identical to SEQ ID NOs: 26, or homologues thereof, wherein the amino acid sequence has at least one amino acid variant on position N31; or
f. an amino acid sequence which is at least 65% identical to SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

SEQ ID NO: 33, which has amino acid sequence SPTCIPSGQPCPYNENCCSQSCTFKENETGNTVKRCD, and which is also known as w-hexatoxin-Ar1f, is a native peptide of Atrax robustus. As far as applicants are aware, SEQ ID NO: 33 has not been disclosed as a purposive mutation, nor has it been disclosed in relation with an increased stability at alkaline pH.

The present invention accordingly relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 27 to 29, or homologues thereof, wherein the amino acid sequence has at least one amino acid variant on position N27;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 2, 6, 7, 30 or 32, or homologues thereof, wherein the amino acid sequence has at least one amino acid variant on position N28;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1, 3, 4, 5, 8 to 24 or 31, or homologues thereof, wherein the amino acid sequence has at least one amino acid variant on position N29;
d. an amino acid sequence which is at least 65%, 70%, 75%, 80%, 85%, 90%, 91%, 92%, 93%, 94%, 95%, 96%, 97%, 98% or 99% identical to SEQ ID NO: 25, or homologues thereof, wherein the amino acid sequence has at least one amino acid variant on position N30;
e. an amino acid sequence which is at least 65%, 70%, 75%, 80%, 85%, 90%, 91%, 92%, 93%, 94%, 95%, 96%, 97%, 98% or 99% identical to SEQ ID NOs: 26, or homologues thereof, wherein the amino acid sequence has at least one amino acid variant on position N31; or
f. an amino acid sequence which is at least 65%, 70%, 75%, 80%, 85%, 90%, 91%, 92%, 93%, 94%, 95%, 96%, 97%, 98% or 99% identical to SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

The present invention accordingly relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 27 to 29, wherein the amino acid sequence has at least one amino acid variant on position N27;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 2, 6, 7, 30 or 32, wherein the amino acid sequence has at least one amino acid variant on position N28;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1, 3, 4, 5, 8 to 24 or 31, wherein the amino acid sequence has at least one amino acid variant on position N29;
d. an amino acid sequence which is at least 65%, 70%, 75%, 80%, 85%, 90%, 91%, 92%, 93%, 94%, 95%, 96%, 97%, 98% or 99% identical to SEQ ID NO: 25, wherein the amino acid sequence has at least one amino acid variant on position N30;
e. an amino acid sequence which is at least 65%, 70%, 75%, 80%, 85%, 90%, 91%, 92%, 93%, 94%, 95%, 96%, 97%, 98% or 99% identical to SEQ ID NOs: 26, wherein the amino acid sequence has at least one amino acid variant on position N31; or
f. an amino acid sequence which is at least 65%, 70%, 75%, 80%, 85%, 90%, 91%, 92%, 93%, 94%, 95%, 96%, 97%, 98% or 99% identical to SEQ ID NOs: 34, wherein the amino acid sequence has at least one amino acid variant on position N32;
   each of which substantially retain the biological activity of the hexatoxin; with the proviso that the peptide does not comprise the amino acid sequence of SEQ ID NO: 33.

The present invention accordingly relates to a hexatoxin peptide variant comprising
a. an amino acid sequence of any one of SEQ ID NOs: 27 to 29, or homologues thereof, wherein the amino acid sequence has at least one amino acid variant on position N27;
b. an amino acid sequence of any one of SEQ ID NOs: 2, 6, 7, 30 or 32, or homologues thereof, wherein the amino acid sequence has at least one amino acid variant on position N28;
c. an amino acid sequence of any one of SEQ ID NOs: 1, 3, 4, 5, 8 to 24 or 31, or homologues thereof, wherein the amino acid sequence has at least one amino acid variant on position N29;
d. an amino acid sequence of SEQ ID NO: 25, or homologues thereof, wherein the amino acid sequence has at least one amino acid variant on position N30;
e. an amino acid sequence of SEQ ID NOs: 26, or homologues thereof, wherein the amino acid sequence has at least one amino acid variant on position N31; or
f. an amino acid sequence of SEQ ID NOs: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 2, 6 or 7, or homologues thereof, wherein the amino acid sequence has at least one amino acid variant on position N28;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1, 3, 4, 5 or 8 to 24, or homologues thereof, wherein the amino acid sequence has at least one amino acid variant on position N29;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 26, or homologues thereof, wherein the amino acid sequence has at least one amino acid variant on position N31; or
d. an amino acid sequence which is at least 65%, 70%, 75%, 80%, 85%, 90%, 91%, 92%, 93%, 94%, 95%, 96%, 97%, 98% or 99% identical to SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 2, 6 or 7, wherein the amino acid sequence has at least one amino acid variant on position N28;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1, 3, 4, 5 or 8 to 24, wherein the amino acid sequence has at least one amino acid variant on position N29;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 26, wherein the amino acid sequence has at least one amino acid variant on position N31; or
d. an amino acid sequence which is at least 65%, 70%, 75%, 80%, 85%, 90%, 91%, 92%, 93%, 94%, 95%, 96%, 97%, 98% or 99% identical to SEQ ID NO: 34, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence of any one of SEQ ID NOs: 2, 6 or 7, or homologues thereof, wherein the amino acid sequence has at least one amino acid variant on position N28;
b. an amino acid sequence of any one of SEQ ID NOs: 1, 3, 4, 5 or 8 to 24, or homologues thereof, wherein the amino acid sequence has at least one amino acid variant on position N29;
c. an amino acid sequence of any one of SEQ ID NOs: 26, or homologues thereof, wherein the amino acid sequence has at least one amino acid variant on position N31; or
d. an amino acid sequence of SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 27 to 29, or homologues thereof, wherein the amino acid sequence has at least one amino acid variant on position N27;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 30 or 32, or homologues thereof, wherein the amino acid sequence has at least one amino acid variant on position N28;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1 or 31, or homologues thereof, wherein the amino acid sequence has at least one amino acid variant on position N29; or
d. an amino acid sequence which is at least 65%, 70%, 75%, 80%, 85%, 90%, 91%, 92%, 93%, 94%, 95%, 96%, 97%, 98% or 99% identical to SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence which is at least 65%, 70%, 75%, 80%, 85%, 90%, 91%, 92%, 93%, 94%, 95%, 96%, 97%, 98% or 99% identical to any one of SEQ ID NOs: 27 to 29, wherein the amino acid sequence has at least one amino acid variant on position N27;
b. an amino acid sequence which is at least 65%, 70%, 75%, 80%, 85%, 90%, 91%, 92%, 93%, 94%, 95%, 96%, 97%, 98% or 99% identical to any one of SEQ ID NOs: 30 or 32, wherein the amino acid sequence has at least one amino acid variant on position N28;
c. an amino acid sequence which is at least 65%, 70%, 75%, 80%, 85%, 90%, 91%, 92%, 93%, 94%, 95%, 96%, 97%, 98% or 99% identical to any one of SEQ ID NOs: 1 or 31, wherein the amino acid sequence has at least one amino acid variant on position N29; or
d. an amino acid sequence which is at least 65%, 70%, 75%, 80%, 85%, 90%, 91%, 92%, 93%, 94%, 95%, 96%, 97%, 98% or 99% identical to SEQ ID NO: 34, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising
a. an amino acid sequence of any one of SEQ ID NOs: 27 to 29, or homologues thereof, wherein the amino acid sequence has at least one amino acid variant on position N27;
b. an amino acid sequence of any one of SEQ ID NOs: 30 or 32, or homologues thereof, wherein the amino acid sequence has at least one amino acid variant on position N28;
c. an amino acid sequence of any one of SEQ ID NOs: 1 or 31, or homologues thereof, wherein the amino acid sequence has at least one amino acid variant on position N29; or
d. an amino acid sequence of SEQ ID NO: 34, or homologues thereof, wherein the amino acid sequence has at least one amino acid variant on position N32;
   each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 1, or homologues thereof, wherein the amino acid sequence has at least one amino acid variant on position N29; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 1, wherein the amino acid sequence has at least one amino acid variant on position N29; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 1, or homologues thereof, wherein the amino acid sequence has at least one amino acid variant on position N29; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 29, or homologues thereof, wherein the amino acid sequence has at least one amino acid variant on position N27; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 29, wherein the amino acid sequence has at least one amino acid variant on position N27; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 29, or homologues thereof, wherein the amino acid sequence has at least one amino acid variant on position N27; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 34, or homologues thereof, wherein the amino acid sequence has at least one amino acid variant on position N32; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 34, wherein the amino acid sequence has at least one amino acid variant on position N32; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 34, or homologues thereof, wherein the amino acid sequence has at least one amino acid variant on position N32; each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 27 to 29, wherein the amino acid sequence has an amino acid variant on position N27;
b. an amino acid sequence of any one of SEQ ID NOs: 2, 6, 7, 30 or 32, wherein the amino acid sequence has an amino acid variant on position N28; or
c. an amino acid sequence of any one of SEQ ID NOs: 1, 3, 4, 5, 8 to 24 or 31, wherein the amino acid sequence has an amino acid variant on position N29.
d. an amino acid sequence of SEQ ID NO: 25, wherein the amino acid sequence has an amino acid variant on position N30;
e. an amino acid sequence of any one of SEQ ID NOs: 26, wherein the amino acid sequence has an amino acid variant on position N31; or
f. an amino acid sequence of SEQ ID NOs: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 27 to 29, wherein the amino acid sequence has an amino acid variant on position N27;
b. an amino acid sequence of any one of or substantially identical to any one of SEQ ID NOs: 2, 6, 7, 30 or 32, wherein the amino acid sequence has an amino acid variant on position N28; or
c. an amino acid sequence of any one of or substantially identical to any one of SEQ ID NOs: 1, 3, 4, 5, 8 to 24 or 31, wherein the amino acid sequence has an amino acid variant on position N29.
d. an amino acid sequence of or substantially identical to any one of SEQ ID NO: 25, wherein the amino acid sequence has an amino acid variant on position N30;
e. an amino acid sequence of or substantially identical to any one of SEQ ID NOs: 26, wherein the amino acid sequence has an amino acid variant on position N31; or
f. an amino acid sequence of or substantially identical to any one of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 2, 6 or 7, wherein the amino acid sequence has an amino acid variant on position N28; or
b. an amino acid sequence of any one of SEQ ID NOs: 1, 3, 4, 5 or 8 to 24, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 2, 6 or 7, wherein the amino acid sequence has an amino acid variant on position N28; or
b. an amino acid sequence of any one of or substantially identical to any one of SEQ ID NOs: 1, 3, 4, 5 or 8 to 24, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 2, 6 or 7, wherein the amino acid sequence has an amino acid variant on position N28; or
b. an amino acid sequence of any one of SEQ ID NOs: 1, 3, 4, 5 or 8 to 24, wherein the amino acid sequence has an amino acid variant on position N29.
c. an amino acid sequence of SEQ ID NO: 25, wherein the amino acid sequence has an amino acid variant on position N30;
d. an amino acid sequence of any one of SEQ ID NOs: 26, wherein the amino acid sequence has an amino acid variant on position N31; or
e. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 2, 6 or 7, wherein the amino acid sequence has an amino acid variant on position N28; or
b. an amino acid sequence of any one of or substantially identical to any one of SEQ ID NOs: 1, 3, 4, 5 or 8 to 24, wherein the amino acid sequence has an amino acid variant on position N29.
c. an amino acid sequence of or substantially identical to any one of SEQ ID NO: 25, wherein the amino acid sequence has an amino acid variant on position N30;
d. an amino acid sequence of or substantially identical to any one of SEQ ID NOs: 26, wherein the amino acid sequence has an amino acid variant on position N31; or
e. an amino acid sequence of or substantially identical to any one of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 27 to 29, wherein the amino acid sequence has an amino acid variant on position N27;
b. an amino acid sequence of any one of SEQ ID NOs: 2, 6, 7, 30 or 32, wherein the amino acid sequence has an amino acid variant on position N28; or
c. an amino acid sequence of any one of SEQ ID NOs: 1, 3, 4, 5, 8 to 24 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 27 to 29, wherein the amino acid sequence has an amino acid variant on position N27;
b. an amino acid sequence of any one of or substantially identical to any one of SEQ ID NOs: 2, 6, 7, 30 or 32, wherein the amino acid sequence has an amino acid variant on position N28; or
c. an amino acid sequence of any one of or substantially identical to any one of SEQ ID NOs: 1, 3, 4, 5, 8 to 24 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 27 to 29, wherein the amino acid sequence has an amino acid variant on position N27;
b. an amino acid sequence of any one of SEQ ID NOs: 30 or 32, wherein the amino acid sequence has an amino acid variant on position N28; or
c. an amino acid sequence of any one of SEQ ID NOs: 1 or 31, wherein the amino acid sequence has an amino acid variant on position N29.
d. an amino acid sequence of SEQ ID NO: 25, wherein the amino acid sequence has an amino acid variant on position N30;
e. an amino acid sequence of SEQ ID NOs: 26, wherein the amino acid sequence has an amino acid variant on position N31; or
f. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 27 to 29, wherein the amino acid sequence has an amino acid variant on position N27;
b. an amino acid sequence of any one of or substantially identical to any one of SEQ ID NOs: 30 or 32, wherein the amino acid sequence has an amino acid variant on position N28; or
c. an amino acid sequence of any one of or substantially identical to any one of SEQ ID NOs: 1 or 31, wherein the amino acid sequence has an amino acid variant on position N29.
d. an amino acid sequence of or substantially identical to any one of SEQ ID NO: 25, wherein the amino acid sequence has an amino acid variant on position N30;
e. an amino acid sequence of or substantially identical to any one of SEQ ID NOs: 26, wherein the amino acid sequence has an amino acid variant on position N31; or
f. an amino acid sequence of or substantially identical to SEQ ID NOs: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NOs: 27 to 29, wherein the amino acid sequence has an amino acid variant on position N27;
b. an amino acid sequence of any one of SEQ ID NOs: 30 or 32, wherein the amino acid sequence has an amino acid variant on position N28; or
c. an amino acid sequence of any one of SEQ ID NOs: 1 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NOs: 27 to 29, wherein the amino acid sequence has an amino acid variant on position N27;
b. an amino acid sequence of any one of or substantially identical to any one of SEQ ID NOs: 30 or 32, wherein the amino acid sequence has an amino acid variant on position N28; or
c. an amino acid sequence of any one of or substantially identical to any one of SEQ ID NOs: 1 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of SEQ ID NO: 1, wherein the amino acid sequence has an amino acid variant on position N29.
b. an amino acid sequence of SEQ ID NO: 25, wherein the amino acid sequence has an amino acid variant on position N30;
c. an amino acid sequence of any one of SEQ ID NOs: 26, wherein the amino acid sequence has an amino acid variant on position N31; or
d. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence of any one of or substantially identical to any one of SEQ ID NO: 1, wherein the amino acid sequence has an amino acid variant on position N29.
b. an amino acid sequence of or substantially identical to any one of SEQ ID NO: 25, wherein the amino acid sequence has an amino acid variant on position N30;
c. an amino acid sequence of or substantially identical to any one of SEQ ID NOs: 26, wherein the amino acid sequence has an amino acid variant on position N31; or
d. an amino acid sequence of or substantially identical to any one of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 29, wherein the amino acid sequence has an amino acid variant on position N27;
b. an amino acid sequence of any one of SEQ ID NOs: 2 or 30, wherein the amino acid sequence has an amino acid variant on position N28; or
c. an amino acid sequence of any one of SEQ ID NOs: 1, 5, 8, 9, 12 or 31, wherein the amino acid sequence has an amino acid variant on position N29.
d. an amino acid sequence of SEQ ID NO: 25, wherein the amino acid sequence has an amino acid variant on position N30;
e. an amino acid sequence of any one of SEQ ID NOs: 26, wherein the amino acid sequence has an amino acid variant on position N31; or
f. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 29, wherein the amino acid sequence has an amino acid variant on position N27;
b. an amino acid sequence of any one of or substantially identical to any one of SEQ ID NOs: 2 or 30, wherein the amino acid sequence has an amino acid variant on position N28; or
c. an amino acid sequence of any one of or substantially identical to any one of SEQ ID NOs: 1, 5, 8, 9, 12 or 31, wherein the amino acid sequence has an amino acid variant on position N29.
d. an amino acid sequence of or substantially identical to any one of SEQ ID NO: 25, wherein the amino acid sequence has an amino acid variant on position N30;
e. an amino acid sequence of or substantially identical to any one of SEQ ID NOs: 26, wherein the amino acid sequence has an amino acid variant on position N31; or
f. an amino acid sequence of or substantially identical to any one of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 2, wherein the amino acid sequence has an amino acid variant on position N28; or
b. an amino acid sequence of any one of SEQ ID NOs: 1, 5, 8, 9 or 12, wherein the amino acid sequence has an amino acid variant on position N29.
c. an amino acid sequence of SEQ ID NO: 25, wherein the amino acid sequence has an amino acid variant on position N30;
d. an amino acid sequence of any one of SEQ ID NOs: 26, wherein the amino acid sequence has an amino acid variant on position N31; or
e. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 2, wherein the amino acid sequence has an amino acid variant on position N28; or
b. an amino acid sequence of any one of or substantially identical to any one of SEQ ID NOs: 1, 5, 8, 9 or 12, wherein the amino acid sequence has an amino acid variant on position N29.
c. an amino acid sequence of or substantially identical to any one of SEQ ID NO: 25, wherein the amino acid sequence has an amino acid variant on position N30;
d. an amino acid sequence of or substantially identical to any one of SEQ ID NOs: 26, wherein the amino acid sequence has an amino acid variant on position N31; or
e. an amino acid sequence of or substantially identical to any one of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 29, wherein the amino acid sequence has an amino acid variant on position N27;
b. an amino acid sequence of any one of SEQ ID NOs: 2 or 30, wherein the amino acid sequence has an amino acid variant on position N28; or
c. an amino acid sequence of any one of SEQ ID NOs: 1, 5, 9, 12 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 29, wherein the amino acid sequence has an amino acid variant on position N27;
b. an amino acid sequence of any one of or substantially identical to any one of SEQ ID NOs: 2 or 30, wherein the amino acid sequence has an amino acid variant on position N28; or
c. an amino acid sequence of any one of or substantially identical to any one of SEQ ID NOs: 1, 5, 9, 12 or 31, wherein the amino acid sequence has an amino acid variant on position N29;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 2, wherein the amino acid sequence has an amino acid variant on position N28; or
b. an amino acid sequence of any one of SEQ ID NOs: 1, 5, 8, 9 or 12, wherein the amino acid sequence has an amino acid variant on position N29;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 2, wherein the amino acid sequence has an amino acid variant on position N28; or
b. an amino acid sequence of any one of or substantially identical to any one of SEQ ID NOs: 1, 5, 8, 9 or 12, wherein the amino acid sequence has an amino acid variant on position N29;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 29, wherein the amino acid sequence has an amino acid variant on position N27;
b. an amino acid sequence of any one of SEQ ID NO: 2, wherein the amino acid sequence has an amino acid variant on position N28; or
c. an amino acid sequence of any one of SEQ ID NOs: 1, 5, 8 or 31, wherein the amino acid sequence has an amino acid variant on position N29.
d. an amino acid sequence of SEQ ID NO: 25, wherein the amino acid sequence has an amino acid variant on position N30;
e. an amino acid sequence of any one of SEQ ID NOs: 26, wherein the amino acid sequence has an amino acid variant on position N31; or
f. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 29, wherein the amino acid sequence has an amino acid variant on position N27;
b. an amino acid sequence of any one of or substantially identical to any one of SEQ ID NO: 2, wherein the amino acid sequence has an amino acid variant on position N28; or
c. an amino acid sequence of any one of or substantially identical to any one of SEQ ID NOs: 1, 5, 8 or 31, wherein the amino acid sequence has an amino acid variant on position N29.
d. an amino acid sequence of or substantially identical to SEQ ID NO: 25, wherein the amino acid sequence has an amino acid variant on position N30;
e. an amino acid sequence of or substantially identical to any one of SEQ ID NOs: 26, wherein the amino acid sequence has an amino acid variant on position N31; or
f. an amino acid sequence of or substantially identical to SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of SEQ ID NO: 29, wherein the amino acid sequence has an amino acid variant on position N27;
b. an amino acid sequence of any one of SEQ ID NO: 2, wherein the amino acid sequence has an amino acid variant on position N28; or
c. an amino acid sequence of any one of SEQ ID NOs: 1 or 31, wherein the amino acid sequence has an amino acid variant on position N29.
d. an amino acid sequence of SEQ ID NO: 25, wherein the amino acid sequence has an amino acid variant on position N30;
e. an amino acid sequence of any one of SEQ ID NOs: 26, wherein the amino acid sequence has an amino acid variant on position N31; or
f. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to any one of SEQ ID NO: 29, wherein the amino acid sequence has an amino acid variant on position N27;
b. an amino acid sequence of any one of or substantially identical to any one of SEQ ID NO: 2, wherein the amino acid sequence has an amino acid variant on position N28;
c. an amino acid sequence of any one of or substantially identical to any one of SEQ ID NOs: 1 or 31, wherein the amino acid sequence has an amino acid variant on position N29.
d. an amino acid sequence of or substantially identical to SEQ ID NO: 25, wherein the amino acid sequence has an amino acid variant on position N30;
e. an amino acid sequence of or substantially identical to any one of SEQ ID NOs: 26, wherein the amino acid sequence has an amino acid variant on position N31; or
f. an amino acid sequence of or substantially identical to SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of SEQ ID NO: 29, wherein the amino acid sequence has an amino acid variant on position N27;
b. an amino acid sequence of SEQ ID NO: 2, wherein the amino acid sequence has an amino acid variant on position N28;
c. an amino acid sequence of SEQ ID NO: 1, wherein the amino acid sequence has an amino acid variant on position N29.
d. an amino acid sequence of SEQ ID NO: 25, wherein the amino acid sequence has an amino acid variant on position N30; or
e. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence of any one of or substantially identical to SEQ ID NO: 29, wherein the amino acid sequence has an amino acid variant on position N27;
b. an amino acid sequence of any one of or substantially identical to SEQ ID NO: 2, wherein the amino acid sequence has an amino acid variant on position N28; or
c. an amino acid sequence of any one of or substantially identical to SEQ ID NO: 1, wherein the amino acid sequence has an amino acid variant on position N29.
d. an amino acid sequence of or substantially identical to SEQ ID NO: 25, wherein the amino acid sequence has an amino acid variant on position N30; or
e. an amino acid sequence of or substantially identical to SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin variant comprising
a. an amino acid sequence of SEQ ID NO: 2, wherein the amino acid sequence has an amino acid variant on position N28;
b. an amino acid sequence of SEQ ID NO: 1, wherein the amino acid sequence has an amino acid variant on position N29; or
c. an amino acid sequence of SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin variant comprising
a. an amino acid sequence of any one of or substantially identical to SEQ ID NO: 2, wherein the amino acid sequence has an amino acid variant on position N28;
b. an amino acid sequence of any one of or substantially identical to SEQ ID NO: 1, wherein the amino acid sequence has an amino acid variant on position N29; or
c. an amino acid sequence of any one of or substantially identical to SEQ ID NO: 34, wherein the amino acid sequence has an amino acid variant on position N32;
   each of which substantially retain the biological activity of the hexatoxin; with the proviso that the peptide does not comprise the amino acid sequence of SEQ ID NO: 33.

Another embodiment of the invention is a hexatoxin peptide variant comprising an amino acid sequence of any one of SEQ ID NO: 1, wherein the amino acid sequence has an amino acid variant on position N29; each of which substantially retain the biological activity of the hexatoxin; with the proviso that the peptide does not comprise the amino acid sequence of SEQ ID NO: 33.

Another embodiment of the invention is a hexatoxin peptide variant comprising an amino acid sequence of any one of or substantially identical to any one of SEQ ID NO: 1, wherein the amino acid sequence has an amino acid variant on position N29; each of which substantially retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence which is at least 90% identical to any one of SEQ ID NOs: 27 to 29, wherein the amino acid sequence has at least one amino acid variant on position N27;
b. an amino acid sequence which is at least 90% identical to any one of SEQ ID NOs: 2, 6, 7, 30 or 32, wherein the amino acid sequence has at least one amino acid variant on position N28;
c. an amino acid sequence which is at least 90% identical to any one of SEQ ID NOs: 1, 3, 4, 5, 8 to 24 or 31, wherein the amino acid sequence has at least one amino acid variant on position N29;
d. an amino acid sequence which is at least 90% identical to SEQ ID NO: 25, wherein the amino acid sequence has at least one amino acid variant on position N30;
e. an amino acid sequence which is at least 90% identical to SEQ ID NO: 26, wherein the amino acid sequence has at least one amino acid variant on position N31; or
f. an amino acid sequence which is at least 90% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 85% identical to any one of SEQ ID NOs: 27 to 29, wherein the amino acid sequence has at least one amino acid variant on position N27;
b. an amino acid sequence which is at least 85% identical to any one of SEQ ID NOs: 2, 6, 7, 30 or 32, wherein the amino acid sequence has at least one amino acid variant on position N28;
c. an amino acid sequence which is at least 85% identical to any one of SEQ ID NOs: 1, 3, 4, 5, 8 to 24 or 31, wherein the amino acid sequence has at least one amino acid variant on position N29;
d. an amino acid sequence which is at least 85% identical to SEQ ID NO: 25, wherein the amino acid sequence has at least one amino acid variant on position N30;
e. an amino acid sequence which is at least 85% identical to SEQ ID NO: 26, wherein the amino acid sequence has at least one amino acid variant on position N31; or
f. an amino acid sequence which is at least 85% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 92% identical to any one of SEQ ID NOs: 27 to 29, wherein the amino acid sequence has at least one amino acid variant on position N27;
b. an amino acid sequence which is at least 92% identical to any one of SEQ ID NOs: 2, 6, 7, 30 or 32, wherein the amino acid sequence has at least one amino acid variant on position N28;
c. an amino acid sequence which is at least 92% identical to any one of SEQ ID NOs: 1, 3, 4, 5, 8 to 24 or 31, wherein the amino acid sequence has at least one amino acid variant on position N29;
d. an amino acid sequence which is at least 92% identical to SEQ ID NO: 25, wherein the amino acid sequence has at least one amino acid variant on position N30;
e. an amino acid sequence which is at least 92% identical to SEQ ID NO: 26, wherein the amino acid sequence has at least one amino acid variant on position N31; or
f. an amino acid sequence which is at least 92% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 94% identical to any one of SEQ ID NOs: 27 to 29, wherein the amino acid sequence has at least one amino acid variant on position N27;
b. an amino acid sequence which is at least 94% identical to any one of SEQ ID NOs: 2, 6, 7, 30 or 32, wherein the amino acid sequence has at least one amino acid variant on position N28;
c. an amino acid sequence which is at least 94% identical to any one of SEQ ID NOs: 1, 3, 4, 5, 8 to 24 or 31, wherein the amino acid sequence has at least one amino acid variant on position N29;
d. an amino acid sequence which is at least 94% identical to SEQ ID NO: 25, wherein the amino acid sequence has at least one amino acid variant on position N30;
e. an amino acid sequence which is at least 94% identical to SEQ ID NO: 26, wherein the amino acid sequence has at least one amino acid variant on position N31; or
f. an amino acid sequence which is at least 94% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 96% identical to any one of SEQ ID NOs: 27 to 29, wherein the amino acid sequence has at least one amino acid variant on position N27;
b. an amino acid sequence which is at least 96% identical to any one of SEQ ID NOs: 2, 6, 7, 30 or 32, wherein the amino acid sequence has at least one amino acid variant on position N28;
c. an amino acid sequence which is at least 96% identical to any one of SEQ ID NOs: 1, 3, 4, 5, 8 to 24 or 31, wherein the amino acid sequence has at least one amino acid variant on position N29;
d. an amino acid sequence which is at least 96% identical to SEQ ID NO: 25, wherein the amino acid sequence has at least one amino acid variant on position N30;
e. an amino acid sequence which is at least 96% identical to SEQ ID NO: 26, wherein the amino acid sequence has at least one amino acid variant on position N31; or
f. an amino acid sequence which is at least 96% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 98% identical to any one of SEQ ID NOs: 27 to 29, wherein the amino acid sequence has at least one amino acid variant on position N27;
b. an amino acid sequence which is at least 98% identical to any one of SEQ ID NOs: 2, 6, 7, 30 or 32, wherein the amino acid sequence has at least one amino acid variant on position N28;
c. an amino acid sequence which is at least 98% identical to any one of SEQ ID NOs: 1, 3, 4, 5, 8 to 24 or 31, wherein the amino acid sequence has at least one amino acid variant on position N29;
d. an amino acid sequence which is at least 98% identical to SEQ ID NO: 25, wherein the amino acid sequence has at least one amino acid variant on position N30;
e. an amino acid sequence which is at least 98% identical to SEQ ID NO: 26, wherein the amino acid sequence has at least one amino acid variant on position N31; or
f. an amino acid sequence which is at least 98% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to any one of SEQ ID NOs: 27 to 29, wherein the amino acid sequence has at least one amino acid variant on position N27; each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to any one of SEQ ID NOs: 2, 6, 7, 30 or 32, wherein the amino acid sequence has at least one amino acid variant on position N28; each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to any one of SEQ ID NOs: 1, 3, 4, 5, 8 to 24 or 31, wherein the amino acid sequence has at least one amino acid variant on position N29; each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to SEQ ID NO: 25, wherein the amino acid sequence has at least one amino acid variant on position N30; each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to SEQ ID NO: 26, wherein the amino acid sequence has at least one amino acid variant on position N31; each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to SEQ ID NO: 34, wherein the amino acid sequence has at least one amino acid variant on position N32; each of which retain the biological activity of the hexatoxin; with the proviso that the peptide does not comprise the amino acid sequence of SEQ ID NO: 33.

Yet another embodiment of the invention is a hexatoxin peptide variant comprising
a. an amino acid sequence which is at least 90% identical to SEQ ID NO: 29, wherein the amino acid sequence has at least one amino acid variant on position N27;
b. an amino acid sequence which is at least 90% identical to SEQ ID NO: 1, wherein the amino acid sequence has at least one amino acid variant on position N29;
c. an amino acid sequence which is at least 90% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 85% identical to SEQ ID NO: 29, wherein the amino acid sequence has at least one amino acid variant on position N27;
b. an amino acid sequence which is at least 85% identical to SEQ ID NO: 1, wherein the amino acid sequence has at least one amino acid variant on position N29;
c. an amino acid sequence which is at least 85% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 92% identical to SEQ ID NO: 29, wherein the amino acid sequence has at least one amino acid variant on position N27;
b. an amino acid sequence which is at least 92% identical to SEQ ID NO: 1, wherein the amino acid sequence has at least one amino acid variant on position N29;
c. an amino acid sequence which is at least 92% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 94% identical to SEQ ID NO: 29, wherein the amino acid sequence has at least one amino acid variant on position N27;
b. an amino acid sequence which is at least 94% identical to SEQ ID NO: 1, wherein the amino acid sequence has at least one amino acid variant on position N29;
c. an amino acid sequence which is at least 94% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 96% identical to SEQ ID NO: 29, wherein the amino acid sequence has at least one amino acid variant on position N27;
b. an amino acid sequence which is at least 96% identical to SEQ ID NO: 1, wherein the amino acid sequence has at least one amino acid variant on position N29;
c. an amino acid sequence which is at least 96% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Another embodiment of the invention is a hexatoxin peptide variant comprising
a. an amino acid sequence which is at least 98% identical to SEQ ID NO: 29, wherein the amino acid sequence has at least one amino acid variant on position N27;
b. an amino acid sequence which is at least 98% identical to SEQ ID NO: 1, wherein the amino acid sequence has at least one amino acid variant on position N29;
c. an amino acid sequence which is at least 98% identical to SEQ ID NO: 34, wherein the amino acid sequence has at least one amino acid variant on position N32;
   each of which retain the biological activity of the hexatoxin; with the proviso that the peptide does not comprise the amino acid sequence of SEQ ID NO: 33.

Yet another embodiment of the invention is a hexatoxin peptide variant comprising

an amino acid sequence which is at least 90% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 85% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 92% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 94% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 96% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

Another embodiment of the invention is a hexatoxin peptide variant comprising

an amino acid sequence which is at least 98% identical to SEQ ID NO: 1, wherein the amino acid sequence has at least one amino acid variant on position N29; each of which retain the biological activity of the hexatoxin; with the proviso that the peptide does not comprise the amino acid sequence of SEQ ID NO: 33.

In addition, an embodiment of the invention is to provide a method for increasing stability of a hexatoxin peptide, comprising introducing an amino acid variant on position N27 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 27 to 29, or homologues thereof, on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 2, 6, 7, 30 or 32, or homologues thereof, on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1, 3, 4, 5, 8 to 24 or 31, or homologues thereof, on position N30 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 25, or homologues thereof, on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 26, or homologues thereof, or on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34, or homologues thereof.

An embodiment of the invention is to provide a method for increasing stability of a hexatoxin peptide, comprising introducing an amino acid variant on position N27 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 27 to 29, on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 2, 6, 7, 30 or 32, on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1, 3, 4, 5, 8 to 24 or 31, on position N30 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 25, on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 26, on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34.

An embodiment of the invention is to provide a method for increasing stability of a hexatoxin peptide, comprising introducing an amino acid variant on position N27 into an amino acid sequence comprising any one of SEQ ID NOs: 27 to 29, or homologues thereof, on position N28 into an amino acid sequence comprising any one of SEQ ID NOs: 2, 6, 7, 30 or 32, or homologues thereof, on position N29 into an amino acid sequence comprising any one of SEQ ID NOs: 1, 3, 4, 5, 8 to 24 or 31, or homologues thereof, on position N30 into an amino acid sequence comprising SEQ ID NO: 25, or homologues thereof, on position N31 into an amino acid sequence comprising any one of SEQ ID NOs: 26, or homologues thereof, or on position N32 into an amino acid sequence comprising SEQ ID NO: 34, or homologues thereof.

In another embodiment the present invention relates to a method for increasing stability of a hexatoxin peptide, comprising introducing an amino acid variant on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 2, 6 or 7, or homologues thereof, or on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1, 3, 4, 5 or 8 to 24, or homologues thereof, on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 26, or homologues thereof, or on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34, or homologues thereof.

In another embodiment the present invention relates to a method for increasing stability of a hexatoxin peptide, comprising introducing an amino acid variant on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 2, 6 or 7, or on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1, 3, 4, 5 or 8 to 24, on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 26, or on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34.

In another embodiment the present invention relates to a method for increasing stability of a hexatoxin peptide, comprising introducing an amino acid variant on position N28 into an amino acid sequence comprising any one of SEQ ID NOs: 2, 6 or 7, or homologues thereof, or on position N29 into an amino acid sequence comprising any one of SEQ ID NOs: 1, 3, 4, 5 or 8 to 24, or homologues thereof, on position N31 into an amino acid sequence comprising SEQ ID NO: 26, or homologues thereof, or on position N32 into an amino acid sequence comprising SEQ ID NO: 34, or homologues thereof.

In another embodiment the present invention relates to a method for increasing stability of a hexatoxin peptide, comprising introducing an amino acid variant on position N27 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 27 to 29, or homologues thereof, on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 30 or 32, or homologues thereof, or on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1 or 31, or homologues thereof.

In another embodiment the present invention relates to a method for increasing stability of a hexatoxin peptide, comprising introducing an amino acid variant on position N27 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 27 to 29, on position N28 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 30 or 32, or on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising any one of SEQ ID NOs: 1 or 31.

In another embodiment the present invention relates to a method for increasing stability of a hexatoxin peptide, comprising introducing an amino acid variant on position N27 into an amino acid sequence comprising any one of SEQ ID NOs: 27 to 29, or homologues thereof, on position N28 into an amino acid sequence comprising any one of SEQ ID NOs: 30 or 32, or homologues thereof, or on position N29 into an amino acid sequence comprising any one of SEQ ID NOs: 1 or 31, or homologues thereof.

In another embodiment the present invention relates to a method for increasing stability of a hexatoxin peptide, comprising introducing an amino acid variant on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 1, or homologues thereof.

In another embodiment the present invention relates to a method for increasing stability of a hexatoxin peptide, comprising introducing an amino acid variant on position N29 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 1.

In another embodiment the present invention relates to a method for increasing stability of a hexatoxin peptide, comprising introducing an amino acid variant on position N29 into an amino acid sequence comprising SEQ ID NO: 1, or homologues thereof.

In another embodiment the present invention relates to a method for increasing stability of a hexatoxin peptide, comprising introducing an amino acid variant on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 26, or homologues thereof.

In another embodiment the present invention relates to a method for increasing stability of a hexatoxin peptide, comprising introducing an amino acid variant on position N31 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 26.

In another embodiment the present invention relates to a method for increasing stability of a hexatoxin peptide, comprising introducing an amino acid variant on position N31 into an amino acid sequence comprising SEQ ID NO: 26, or homologues thereof.

In another embodiment the present invention relates to a method for increasing stability of a hexatoxin peptide, comprising introducing an amino acid variant on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34, or homologues thereof.

In another embodiment the present invention relates to a method for increasing stability of a hexatoxin peptide, comprising introducing an amino acid variant on position N32 into an amino acid sequence that is at least 65%, 70%, 75%, 80%, 85%, 90%, 91%, 92%, 93%, 94%, 95%, 96%, 97%, 98% or 99% identical to an amino acid sequence comprising SEQ ID NO: 34.

In another embodiment the present invention relates to a method for increasing stability of a hexatoxin peptide, comprising introducing an amino acid variant on position N32 into an amino acid sequence comprising SEQ ID NO: 34, or homologues thereof.

Another embodiment of the invention is a method for increasing stability of a hexatoxin peptide, comprising introducing an amino acid variant on position N27 into an amino acid sequence comprising any one of SEQ ID NOs: 27 to 29, on position N28 into an amino acid sequence comprising any one of SEQ ID NOs: 2, 6, 7, 30 or 32, on position N29 into an amino acid sequence comprising any one of SEQ ID NOs: 1, 3, 4, 5, 8 to 24 or 31, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Another embodiment of the invention is a method for increasing stability of a hexatoxin peptide, comprising introducing an amino acid variant on position N28 into an amino acid sequence comprising any one of SEQ ID NOs: 2, 6 or 7, or on position N29 into an amino acid sequence comprising any one of SEQ ID NOs: 1, 3, 4, 5 or 8 to 24, on position N31 into an amino acid sequence comprising SEQ ID NO: 26, or on position N32 into an amino acid sequence comprising SEQ ID NO: 34.

Another embodiment of the invention is a method for increasing stability of a hexatoxin peptide, comprising introducing an amino acid variant on position N28 into an amino acid sequence comprising any one of SEQ ID NOs: 2, 6 or 7, on position N29 into an amino acid sequence comprising any one of SEQ ID NOs: 1, 3, 4, 5 or 8 to 24, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Another embodiment of the invention is a method for increasing stability of a hexatoxin peptide, comprising introducing an amino acid variant on position N27 into an amino acid sequence comprising any one of SEQ ID NOs: 27 to 29, on position N28 into an amino acid sequence comprising any one of SEQ ID NOs: 2, 6, 7, 30 or 32 or on position N29 into an amino acid sequence comprising any one of SEQ ID NOs: 1, 3, 4, 5, 8 to 24 or 31.

Another embodiment of the invention is a method for increasing stability of a hexatoxin peptide, comprising introducing an amino acid variant on position N27 into an amino acid sequence comprising any one of SEQ ID NOs: 27 to 29, on position N28 into an amino acid sequence comprising any one of SEQ ID NOs: 30 or 32, on position N29 into an amino acid sequence comprising any one of SEQ ID NOs: 1 or 31, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Another embodiment of the invention is a method for increasing stability of a hexatoxin peptide, comprising introducing an amino acid variant on position N27 into an amino acid sequence comprising any one of SEQ ID NOs: 27 to 29, on position N28 into an amino acid sequence comprising any one of SEQ ID NOs: 30 or 32 or on position N29 into an amino acid sequence comprising any one of SEQ ID NOs: 1 or 31.

Another embodiment of the invention is a method for increasing stability of a hexatoxin peptide, comprising introducing an amino acid variant on position N29 into an amino acid sequence comprising SEQ ID NO: 1, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Yet another embodiment of the invention is a method for increasing stability of a hexatoxin peptide, comprising introducing an amino acid variant on position N27 into an amino acid sequence comprising SEQ ID NO: 29, on position N28 into an amino acid sequence comprising any one of SEQ ID NOs: 2 or 30, on position N29 into an amino acid sequence comprising any one of SEQ ID NOs: 1, 5, 8, 9, 12 or 31, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Yet another embodiment of the invention a method for increasing stability of a hexatoxin peptide, comprising introducing an amino acid variant on position N28 into an amino acid sequence comprising SEQ ID NO: 2, on position N29 into an amino acid sequence comprising any one of SEQ ID NOs: 1, 5, 8, 9 or 12, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Yet another embodiment of the invention is a method for increasing stability of a hexatoxin peptide, comprising introducing an amino acid variant on position N27 into an amino acid sequence comprising SEQ ID NO: 29, on position N28 into an amino acid sequence comprising any one of SEQ ID NOs: 2 or 30 or on position N29 into an amino acid sequence comprising any one of SEQ ID NOs: 1, 5, 8, 9, 12 or 31.

Yet another embodiment of the invention is a method for increasing stability of a hexatoxin peptide, comprising introducing an amino acid variant on position N28 into an amino acid sequence comprising SEQ ID NO: 2 or on position N29 into an amino acid sequence comprising any one of SEQ ID NOs: 1, 5, 8, 9 or 12.

Yet another embodiment of the invention a method for increasing stability of a hexatoxin peptide, comprising introducing an amino acid variant on position N27 into an amino acid sequence comprising SEQ ID NO: 29, on position N28 into an amino acid sequence comprising SEQ ID NO: 2, on position N29 into an amino acid sequence comprising any one of SEQ ID NOs: 1, 5, 8 or 31, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Yet another embodiment of the invention is a method for increasing stability of a hexatoxin peptide, comprising introducing an amino acid variant on position N27 into an amino acid sequence comprising SEQ ID NO: 29, on position N28 into an amino acid sequence comprising SEQ ID NO: 2, on position N29 into an amino acid sequence comprising any one of SEQ ID NOs: 1 or 31, on position N30 into an amino acid sequence comprising SEQ ID NO: 25, on position N31 into an amino acid sequence comprising any one of SEQ ID NOs: 26, or on position N32 into an amino acid sequence comprising SEQ ID NO: 34.

Yet another embodiment of the invention is a method for increasing stability of a hexatoxin peptide, comprising introducing an amino acid variant on position N27 into an amino acid sequence comprising SEQ ID NO: 29, on position N28 into an amino acid sequence comprising SEQ ID NO: 2, on position N29 into an amino acid sequence comprising SEQ ID NO: 1 or on position N30 into an amino acid sequence comprising SEQ ID NO: 25, on position N31 into an amino acid sequence comprising SEQ ID NO: 26, or on position N32 into an amino acid sequence comprising SEQ ID NO: 34.

Another embodiment of the invention is a method for increasing stability of a hexatoxin peptide, comprising introducing an amino acid variant on position N28 into an amino acid sequence comprising SEQ ID NO: 2, or on position N29 into an amino acid sequence comprising SEQ ID NO: 1, or on position N31 into an amino acid sequence comprising SEQ ID NO: 26. Another embodiment of the invention is a method for increasing stability of a hexatoxin peptide, comprising introducing an amino acid variant on position N29 into an amino acid sequence comprising SEQ ID NO: 1, or on position N31 into an amino acid sequence comprising SEQ ID NO: 26. Another embodiment of the invention is a method for increasing stability of a hexatoxin peptide, comprising introducing an amino acid variant on position N32 into an amino acid sequence comprising SEQ ID NO: 34.

Yet another embodiment of the invention is to provide a method for increasing stability of a hexatoxin peptide, comprising introducing an amino acid variant on position N27 into an amino acid sequence that is at least 90% identical to an amino acid sequence comprising any one of SEQ ID NOs: 27 to 29, on position N28 into an amino acid sequence that is at least 90% identical to an amino acid sequence comprising any one of SEQ ID NOs: 2, 6, 7, 30 or 32, on position N29 into an amino acid sequence that is at least 90% identical to an amino acid sequence comprising any one of SEQ ID NOs: 1, 3, 4, 5, 8 to 24 or 31, on position N30 into an amino acid sequence that is at least 90% identical to an amino acid sequence comprising SEQ ID NO: 25, on position N31 into an amino acid sequence that is at least 90% identical to an amino acid sequence comprising SEQ ID NO: 26, or on position N32 into an amino acid sequence that is at least 90% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 85% identical to an amino acid sequence comprising any one of SEQ ID NOs: 27 to 29, on position N28 into an amino acid sequence that is at least 85% identical to an amino acid sequence comprising any one of SEQ ID NOs: 2, 6, 7, 30 or 32, on position N29 into an amino acid sequence that is at least 85% identical to an amino acid sequence comprising any one of SEQ ID NOs: 1, 3, 4, 5, 8 to 24 or 31, on position N30 into an amino acid sequence that is at least 85% identical to an amino acid sequence comprising SEQ ID NO: 25, on position N31 into an amino acid sequence that is at least 85% identical to an amino acid sequence comprising SEQ ID NO: 26, or on position N32 into an amino acid sequence that is at least 85% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 92% identical to an amino acid sequence comprising any one of SEQ ID NOs: 27 to 29, on position N28 into an amino acid sequence that is at least 92% identical to an amino acid sequence comprising any one of SEQ ID NOs: 2, 6, 7, 30 or 32, on position N29 into an amino acid sequence that is at least 92% identical to an amino acid sequence comprising any one of SEQ ID NOs: 1, 3, 4, 5, 8 to 24 or 31, on position N30 into an amino acid sequence that is at least 92% identical to an amino acid sequence comprising SEQ ID NO: 25, on position N31 into an amino acid sequence that is at least 92% identical to an amino acid sequence comprising SEQ ID NO: 26, or on position N32 into an amino acid sequence that is at least 92% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 94% identical to an amino acid sequence comprising any one of SEQ ID NOs: 27 to 29, on position N28 into an amino acid sequence that is at least 94% identical to an amino acid sequence comprising any one of SEQ ID NOs: 2, 6, 7, 30 or 32, on position N29 into an amino acid sequence that is at least 94% identical to an amino acid sequence comprising any one of SEQ ID NOs: 1, 3, 4, 5, 8 to 24 or 31, on position N30 into an amino acid sequence that is at least 94% identical to an amino acid sequence comprising SEQ ID NO: 25, on position N31 into an amino acid sequence that is at least 94% identical to an amino acid sequence comprising SEQ ID NO: 26, or on position N32 into an amino acid sequence that is at least 94% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 96% identical to an amino acid sequence comprising any one of SEQ ID NOs: 27 to 29, on position N28 into an amino acid sequence that is at least 96% identical to an amino acid sequence comprising any one of SEQ ID NOs: 2, 6, 7, 30 or 32, on position N29 into an amino acid sequence that is at least 96% identical to an amino acid sequence comprising any one of SEQ ID NOs: 1, 3, 4, 5, 8 to 24 or 31, on position N30 into an amino acid sequence that is at least 96% identical to an amino acid sequence comprising SEQ ID NO: 25, on position N31 into an amino acid sequence that is at least 96% identical to an amino acid sequence comprising SEQ ID NO: 26, or on position N32 into an amino acid sequence that is at least 96% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 98% identical to an amino acid sequence comprising any one of SEQ ID NOs: 27 to 29, on position N28 into an amino acid sequence that is at least 98% identical to an amino acid sequence comprising any one of SEQ ID NOs: 2, 6, 7, 30 or 32, on position N29 into an amino acid sequence that is at least 98% identical to an amino acid sequence comprising any one of SEQ ID NOs: 1, 3, 4, 5, 8 to 24 or 31, on position N30 into an amino acid sequence that is at least 98% identical to an amino acid sequence comprising SEQ ID NO: 25, on position N31 into an amino acid sequence that is at least 98% identical to an amino acid sequence comprising SEQ ID NO: 26, or on position N32 into an amino acid sequence that is at least 98% identical to an amino acid sequence comprising SEQ ID NO: 34.

Yet another embodiment of the invention is to provide a method for increasing stability of a hexatoxin peptide, comprising introducing an amino acid variant on position N27 into an amino acid sequence that is at least 90% identical to an amino acid sequence comprising any one of SEQ ID NOs: 27 to 29.

Another embodiment of the invention is to provide a method for increasing stability of a hexatoxin peptide, comprising introducing an amino acid variant on position N27 into an amino acid sequence that is at least 85% identical to an amino acid sequence comprising any one of SEQ ID NOs: 27 to 29.

Another embodiment of the invention is to provide a method for increasing stability of a hexatoxin peptide, comprising introducing an amino acid variant on position N27 into an amino acid sequence that is at least 92% identical to an amino acid sequence comprising any one of SEQ ID NOs: 27 to 29.

Another embodiment of the invention is to provide a method for increasing stability of a hexatoxin peptide, comprising introducing an amino acid variant on position N27 into an amino acid sequence that is at least 94% identical to an amino acid sequence comprising any one of SEQ ID NOs: 27 to 29.

Another embodiment of the invention is to provide a method for increasing stability of a hexatoxin peptide, comprising introducing an amino acid variant on position N27 into an amino acid sequence that is at least 96% identical to an amino acid sequence comprising any one of SEQ ID NOs: 27 to 29.

Another embodiment of the invention is to provide a method for increasing stability of a hexatoxin peptide, comprising introducing an amino acid variant on position N27 into an amino acid sequence that is at least 98% identical to an amino acid sequence comprising any one of SEQ ID NOs: 27 to 29.

Yet another embodiment of the invention is to provide a method for increasing stability of a hexatoxin peptide, comprising introducing an amino acid variant on position N28 into an amino acid sequence that is at least 90% identical to an amino acid sequence comprising any one of SEQ ID NOs: 2, 6, 7, 30 or 32.

Another embodiment of the invention is to provide a method for increasing stability of a hexatoxin peptide, comprising introducing an amino acid variant on position N28 into an amino acid sequence that is at least 85% identical to an amino acid sequence comprising any one of SEQ ID NOs: 2, 6, 7, 30 or 32.

Another embodiment of the invention is to provide a method for increasing stability of a hexatoxin peptide, comprising introducing an amino acid variant on position N28 into an amino acid sequence that is at least 92% identical to an amino acid sequence comprising any one of SEQ ID NOs: 2, 6, 7, 30 or 32.

Another embodiment of the invention is to provide a method for increasing stability of a hexatoxin peptide, comprising introducing an amino acid variant on position N28 into an amino acid sequence that is at least 94% identical to an amino acid sequence comprising any one of SEQ ID NOs: 2, 6, 7, 30 or 32.

Another embodiment of the invention is to provide a method for increasing stability of a hexatoxin peptide, comprising introducing an amino acid variant on position N28 into an amino acid sequence that is at least 96% identical to an amino acid sequence comprising any one of SEQ ID NOs: 2, 6, 7, 30 or 32.

Another embodiment of the invention is to provide a method for increasing stability of a hexatoxin peptide, comprising introducing an amino acid variant on position N28 into an amino acid sequence that is at least 98% identical to an amino acid sequence comprising any one of SEQ ID NOs: 2, 6, 7, 30 or 32.

Yet another embodiment of the invention is to provide a method for increasing stability of a hexatoxin peptide, comprising introducing an amino acid variant on position N29 into an amino acid sequence that is at least 90% identical to an amino acid sequence comprising any one of SEQ ID NOs: 1, 3, 4, 5, 8 to 24 or 31.

Another embodiment of the invention is to provide a method for increasing stability of a hexatoxin peptide, comprising introducing an amino acid variant on position N29 into an amino acid sequence that is at least 85% identical to an amino acid sequence comprising any one of SEQ ID NOs: 1, 3, 4, 5, 8 to 24 or 31.

Another embodiment of the invention is to provide a method for increasing stability of a hexatoxin peptide, comprising introducing an amino acid variant on position N29 into an amino acid sequence that is at least 92% identical to an amino acid sequence comprising any one of SEQ ID NOs: 1, 3, 4, 5, 8 to 24 or 31.

Another embodiment of the invention is to provide a method for increasing stability of a hexatoxin peptide, comprising introducing an amino acid variant on position N29 into an amino acid sequence that is at least 94% identical to an amino acid sequence comprising any one of SEQ ID NOs: 1, 3, 4, 5, 8 to 24 or 31.

Another embodiment of the invention is to provide a method for increasing stability of a hexatoxin peptide, comprising introducing an amino acid variant on position N29 into an amino acid sequence that is at least 96% identical to an amino acid sequence comprising any one of SEQ ID NOs: 1, 3, 4, 5, 8 to 24 or 31.

Another embodiment of the invention is to provide a method for increasing stability of a hexatoxin peptide, comprising introducing an amino acid variant on position N29 into an amino acid sequence that is at least 98% identical to an amino acid sequence comprising any one of SEQ ID NOs: 1, 3, 4, 5, 8 to 24 or 31.

Yet another embodiment of the invention is to provide a method for increasing stability of a hexatoxin peptide, comprising introducing an amino acid variant on position N30 into an amino acid sequence that is at least 90% identical to an amino acid sequence comprising SEQ ID NO: 25.

Another embodiment of the invention is to provide a method for increasing stability of a hexatoxin peptide, comprising introducing an amino acid variant on position N30 into an amino acid sequence that is at least 85% identical to an amino acid sequence comprising SEQ ID NO: 25.

Another embodiment of the invention is to provide a method for increasing stability of a hexatoxin peptide, comprising introducing an amino acid variant on position N30 into an amino acid sequence that is at least 92% identical to an amino acid sequence comprising SEQ ID NO: 25.

Another embodiment of the invention is to provide a method for increasing stability of a hexatoxin peptide, comprising introducing an amino acid variant on position N30 into an amino acid sequence that is at least 94% identical to an amino acid sequence comprising SEQ ID NO: 25.

Another embodiment of the invention is to provide a method for increasing stability of a hexatoxin peptide, comprising introducing an amino acid variant on position N30 into an amino acid sequence that is at least 96% identical to an amino acid sequence comprising SEQ ID NO: 25.

Another embodiment of the invention is to provide a method for increasing stability of a hexatoxin peptide, comprising introducing an amino acid variant on position N30 into an amino acid sequence that is at least 98% identical to an amino acid sequence comprising SEQ ID NO: 25.

Yet another embodiment of the invention is to provide a method for increasing stability of a hexatoxin peptide, comprising introducing an amino acid variant on position N31 into an amino acid sequence that is at least 90% identical to an amino acid sequence comprising SEQ ID NO: 26.

Another embodiment of the invention is to provide a method for increasing stability of a hexatoxin peptide, comprising introducing an amino acid variant on position N31 into an amino acid sequence that is at least 85% identical to an amino acid sequence comprising SEQ ID NO: 26.

Another embodiment of the invention is to provide a method for increasing stability of a hexatoxin peptide, comprising introducing an amino acid variant on position N31 into an amino acid sequence that is at least 92% identical to an amino acid sequence comprising SEQ ID NO: 26.

Another embodiment of the invention is to provide a method for increasing stability of a hexatoxin peptide, comprising introducing an amino acid variant on position N31 into an amino acid sequence that is at least 94% identical to an amino acid sequence comprising SEQ ID NO: 26.

Another embodiment of the invention is to provide a method for increasing stability of a hexatoxin peptide, comprising introducing an amino acid variant on position N31 into an amino acid sequence that is at least 96% identical to an amino acid sequence comprising SEQ ID NO: 26.

Another embodiment of the invention is to provide a method for increasing stability of a hexatoxin peptide, comprising introducing an amino acid variant on position N31 into an amino acid sequence that is at least 98% identical to an amino acid sequence comprising SEQ ID NO: 26.

Yet another embodiment of the invention is to provide a method for increasing stability of a hexatoxin peptide, comprising introducing an amino acid variant on position N32 into an amino acid sequence that is at least 90% identical to an amino acid sequence comprising SEQ ID NO: 34.

Another embodiment of the invention is to provide a method for increasing stability of a hexatoxin peptide, comprising introducing an amino acid variant on position N32 into an amino acid sequence that is at least 85% identical to an amino acid sequence comprising SEQ ID NO: 34.

Another embodiment of the invention is to provide a method for increasing stability of a hexatoxin peptide, comprising introducing an amino acid variant on position N32 into an amino acid sequence that is at least 92% identical to an amino acid sequence comprising SEQ ID NO: 34.

Another embodiment of the invention is to provide a method for increasing stability of a hexatoxin peptide, comprising introducing an amino acid variant on position N32 into an amino acid sequence that is at least 94% identical to an amino acid sequence comprising SEQ ID NO: 34.

Another embodiment of the invention is to provide a method for increasing stability of a hexatoxin peptide, comprising introducing an amino acid variant on position N32 into an amino acid sequence that is at least 96% identical to an amino acid sequence comprising SEQ ID NO: 34.

Another embodiment of the invention is to provide a method for increasing stability of a hexatoxin peptide, comprising introducing an amino acid variant on position N32 into an amino acid sequence that is at least 98% identical to an amino acid sequence comprising SEQ ID NO: 34.

Yet another embodiment of the invention is to provide a method for increasing stability of a hexatoxin peptide, comprising introducing an amino acid variant on position N27 into an amino acid sequence that is at least 90% identical to an amino acid sequence comprising SEQ ID NO: 29, on position N29 into an amino acid sequence that is at least 90% identical to an amino acid sequence comprising SEQ ID NO: 1, or on position N32 into an amino acid sequence that is at least 90% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 85% identical to an amino acid sequence comprising SEQ ID NO: 29, on position N29 into an amino acid sequence that is at least 85% identical to an amino acid sequence comprising SEQ ID NO: 1, or on position N32 into an amino acid sequence that is at least 85% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 92% identical to an amino acid sequence comprising SEQ ID NO: 29, on position N29 into an amino acid sequence that is at least 92% identical to an amino acid sequence comprising SEQ ID NO: 1, or on position N32 into an amino acid sequence that is at least 92% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 94% identical to an amino acid sequence comprising SEQ ID NO: 29, on position N29 into an amino acid sequence that is at least 94% identical to an amino acid sequence comprising SEQ ID NO: 1, or on position N32 into an amino acid sequence that is at least 94% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 96% identical to an amino acid sequence comprising SEQ ID NO: 29, on position N29 into an amino acid sequence that is at least 96% identical to an amino acid sequence comprising SEQ ID NO: 1, or on position N32 into an amino acid sequence that is at least 96% identical to an amino acid sequence comprising SEQ ID NO: 34.

Another embodiment of the invention is to provide a method for increasing stability of a hexatoxin peptide, comprising introducing an amino acid variant on position N27 into an amino acid sequence that is at least 98% identical to an amino acid sequence comprising SEQ ID NO: 29, on position N29 into an amino acid sequence that is at least 98% identical to an amino acid sequence comprising SEQ ID NO: 1, or on position N32 into an amino acid sequence that is at least 98% identical to an amino acid sequence comprising SEQ ID NO: 34.

Yet another embodiment of the invention is to provide a method for increasing stability of a hexatoxin peptide, comprising introducing an amino acid variant on position N29 into an amino acid sequence that is at least 90% identical to an amino acid sequence comprising SEQ ID NO: 1.

Another embodiment of the invention is to provide a method for increasing stability of a hexatoxin peptide, comprising introducing an amino acid variant on position N29 into an amino acid sequence that is at least 85% identical to an amino acid sequence comprising SEQ ID NO: 1.

Another embodiment of the invention is to provide a method for increasing stability of a hexatoxin peptide, comprising introducing an amino acid variant on position N29 into an amino acid sequence that is at least 92% identical to an amino acid sequence comprising SEQ ID NO: 1.

Another embodiment of the invention is to provide a method for increasing stability of a hexatoxin peptide, comprising introducing an amino acid variant on position N29 into an amino acid sequence that is at least 94% identical to an amino acid sequence comprising SEQ ID NO: 1.

Another embodiment of the invention is to provide a method for increasing stability of a hexatoxin peptide, comprising introducing an amino acid variant on position N29 into an amino acid sequence that is at least 96% identical to an amino acid sequence comprising SEQ ID NO: 1.

Another embodiment of the invention is to provide a method for increasing stability of a hexatoxin peptide, comprising introducing an amino acid variant on position N29 into an amino acid sequence that is at least 98% identical to an amino acid sequence comprising SEQ ID NO: 1.

Position N27 of an amino acid sequence comprising any one of SEQ ID NOs: 27 to 29, position N28 of an amino acid sequence comprising any one of SEQ ID NOs: 2, 6, 7, 30 or 32, position N29 of an amino acid sequence comprising any one of SEQ ID NOs: 1, 3, 4, 5, 8 to 24 or 31, position N30 of an amino acid sequence comprising SEQ ID NO: 25, position N31 of an amino acid sequence comprising any one of SEQ ID NOs: 26 and position N32 of an amino acid sequence comprising SEQ ID NO: 34 have been found to be critical for the stability of the peptide at an alkaline pH-value.

Alkaline pH-value is herein understood to mean a pH value of greater than 7. Preferably a pH value of from 7 to 14. pH is used to specify the acidity or alkalinity of a water-based solution. Hence, the amino acid sequence or peptide is considered to be in a water-based solution when stability is assessed.

Stability is herein understood to mean the chemical stability of a substance, which is understood to mean the degree of reactivity of a substance. Hence, a low reactivity of a substance results in a high stability of the substance.

Water-based solution is herein understood to mean a solution wherein the solvent is water.

The peptides of the non-variant amino acid sequences described herein have been found to be sensitive to deamidation. Deamidation was found to occur in an aqueous solution when the amide functional group in the side chain of the amino acids asparagine or glutamine was removed or converted to a different functional group by a chemical reaction. Asparagine can be converted to L- or D-succinimidyl, L- or D-aspartyl or L or D-isoaspartyl, while glutamine can be converted to L- or D-glutamyl or L- or D-isoglutamyl. Deamidation is a spontaneous non-enzymatic reaction which occurs frequently and is an undesirable event in the development of industrial applicable peptides. The deamidation reaction can result in an alteration of the primary, secondary and tertiary structure of a peptide, thereby potentially altering its biological function or activity and may lead to degradation of the peptide. Rates of deamidation depend on several factors such as primary sequence, three-dimensional structure, temperature, pH of the solution and other components of the solution such as buffer ions used.

Applicants disclose herein that the hexatoxin peptides according to the invention comprising an amino acid variant on position N27 of an amino acid sequence comprising any one of SEQ ID NOs: 27 to 29, position N28 of an amino acid sequence comprising any one of SEQ ID NOs: 2, 6, 7, 30 or 32, position N29 of an amino acid sequence comprising any one of SEQ ID NOs: 1, 3, 4, 5, 8 to 24 or 31, position N30 of an amino acid sequence comprising SEQ ID NO: 25, position N31 of an amino acid sequence comprising any one of SEQ ID NOs: 26 and position N32 of an amino acid sequence comprising SEQ ID NO: 34 can be changed to other amino acids, thereby increasing the stability of the hexatoxin peptide at an alkaline pH-value while substantially retaining the biological activity of the peptide.

Therefore, the present invention relates to a hexatoxin peptide according to the invention wherein the peptide has increased stability compared to its non-variant amino acid sequence. Preferably, the hexatoxin peptide having increased stability compared to its non-variant amino acid sequence has similar or greater pesticidal activity compared to its non-variant amino acid sequence. Preferably, the hexatoxin peptide having increased stability compared to its non-variant amino acid sequence retains pesticidal activity. Also, the present invention relates to a method for increasing stability of a hexatoxin peptide, wherein the peptide has increased stability compared to its non-variant amino acid sequence.

Similar pesticidal activity is understood to mean a pesticidal activity in a pest feeding assay as described in the examples of this disclosure, wherein one skilled in the art would consider the pesticidal activity of the peptide according to the invention compared to its unsubstituted amino acid sequence to be about the same.

The present invention also relates to a hexatoxin peptide according to the invention having increased stability compared to its non-variant amino acid sequence, wherein the increased stability is in an alkaline medium of greater than pH 7. Also, the present invention relates to a method for increasing stability of a hexatoxin peptide according to the invention having increased stability compared to its non-variant amino acid sequence, wherein the increased stability is in an alkaline medium of greater than pH 7.

Preferably, the alkaline medium is of from pH 7 to pH 14, of from pH 7 to pH 13, of from pH 7 to pH 12, of from pH 7 to pH 11, of from pH 7 to pH 10, of from pH 7 to pH 9, of from pH 7 to pH 8, of from pH 8 to pH 13, of from pH 8 to pH 12, of from pH 8 to pH 11, of from pH 8 to pH 10, of from pH 8 to pH 9, of from pH 9 to pH 11 or of from pH 9 to pH 10.

The stability or the degree of reactivity of the hexatoxin peptide according to the invention is meant to understood with regard to its degree of deamidation. Therefore, an aspect of the current invention is a hexatoxin peptide according to the invention, wherein the toxin peptide has less than 75% deamidation compared to its non-variant amino acid sequence. Also, the present invention relates to a method for increasing stability of a hexatoxin peptide according to the invention, wherein the hexatoxin peptide has less than 75% deamidation compared to its non-variant amino acid sequence. Preferably, the hexatoxin peptide has a deamidation compared to its non-variant amino acid sequence of less than 75%, 70%, 65%, 55%, 50%, 45%, 40%, 35%, 30%, 25%, 20%, 15%, 10%, 5% or 5% or less.

Deamidation of peptides can be determined by methods well-known in the art. Examples of suitable methods are analysis by liquid chromatography-mass spectrometry (LCMS) as for example described by Diepold et al., PLoS ONE, 2012.

The present invention also relates to a hexatoxin peptide according to the invention, wherein the peptide has less than 75%, 70%, 65%, 55%, 50%, 45%, 40%, 35%, 30%, 25%, 20%, 15%, 10%, 5% or 5% or less deamidation compared to its non-variant amino acid sequence, wherein the deamidation is determined after incubation in an alkaline medium of about pH 8 and at 37° C., over a period of 48 hours. Also, the present invention relates to a method for increasing stability of a hexatoxin peptide according to the invention, wherein the peptide has less than 75%, 70%, 65%, 55%, 50%, 45%, 40%, 35%, 30%, 25%, 20%, 15%, 10%, 5% or 5% or less deamidation compared to its non-variant amino acid sequence, wherein the deamidation is determined after incubation in an alkaline medium of about pH 8 and at 37° C. over a period of 48 hours. pH 8 is maintained by a suitable buffer. Preferably, the period is 48, 72, 96 or 168 hours.

The increased stability can be monitored in an alkaline medium in the range of from pH 7 to pH 14, and at a temperature of from 25° C. to 42° C., over a period of at least one day, preferably over a period of at least two days. The alkaline medium is preferably an aqueous buffer. The alkaline medium is preferably in the range of from about pH 8 to about pH 9. Preferably, the temperature is about 25° C. The period is preferably 24, 48, 72, 96, 120, 144, 168, 240, 360 or 480 h. The degree of deamidation can be assessed by mass spectrometry or LCMS as a percentage of predicted total possible deamidation. The degree of stability can be assessed by LCMS as the percentage of remaining parent peak. Examples 3, 4 and 5 describe a suitable experiments incorporating the elements mentioned above.

Variants, functional variants and homologues of the sequences described herein are also encompassed by the embodiments of the present invention. Variants herein are understood to refer to peptides having substantially similar amino acid sequences. Functional variants herein are understood to refer to variants that are biologically active, herein understood to mean that they retain the biological activity of the native peptide sequence, herein understood to mean pesticidal activity as described herein. Homologues herein are understood to mean peptides having sequence similarity to sequences according to the present application, which are suitable for the processes described herein such as pesticidal activity, and which are naturally occurring sequences.

Variants, functional variants and homologues as described herein encompass peptides that are derived from native peptides by substitution, insertion, deletion or addition of at least one amino acid on at least one position in the native peptide. Substitution, insertion or addition of at least one amino acid on at least one position in the native peptide comprises the substitution, insertion or addition by a non-proteinogenic amino acid. Non-proteinogenic amino acids herein are understood to mean amino acids that are not naturally encoded or found in the genetic code of any organism and are not translationally incorporated into proteins. They comprise any organic compound with an amine and a carboxylic acid functional group. Non-limitative examples of these are phenylalanine derivatives such as 4-methyl-phenylalanine and 3,4-dihydroxy-phenylalanine; phenylglycine derivatives such as 4-hydroxy-phenylglycine; tryptophan derivatives such as 6-amino-7-hydroxy-1-tryptophan; methionine derivatives such as nitrilo-l-methionine; alanine derivatives such as adamantane; cysteine derivatives such as penicillamine; asparagine/glutamine derivatives such as cysteine-s-acetamide; lysine derivatives such as 2,3-diaminopropanoic acid; arginine derivatives such as c-gamma-hydroxy arginine; serine/threonine derivatives such as homoserine and phosphothreonine; histidine derivatives such as 2-fluoro-1-histidine and asparagine/glutamine derivatives such as l-2-amino-6-methylene-pimelic acid and 4-fluoro-glutamic acid. More examples are for example illustrated in the SwissSidechain database (http://www.swisssidechain.ch) (Gfeller et al., Nucleic Acids Research, 2013).

Substantially similar amino acid sequences have only a small number of sequence changes, for example in non-conserved residues.

Functional variants also comprise variants that have sequence changes that do not affect function, for example in non-conserved residues.

Changes in a nucleic acid sequence by mutation, substitution, insertion, deletion or addition that lead to changes in the amino acid sequence of the encoded peptide, but without altering its biological activity (i.e. having pesticidal activity), are well-known in the art. These are also known as conservative alterations. For example, a codon for alanine, a hydrophobic amino acid, may be substituted by a codon encoding another less hydrophobic amino acid, such as glycine, or a more hydrophobic amino acid, such as valine, leucine, or isoleucine. Similarly, changes which result in substitution of one negatively charged amino acid for another, such as aspartic acid for glutamic acid, or one positively charged amino acid for another, such as lysine for arginine, can also be expected to produce a functionally equivalent product. Nucleotide changes at the N-terminal and/or C-terminal ends of a non-cyclic peptide are also expected to not alter the biological activity of the non-cyclic peptide.

However, changes in nucleic acid sequences of conserved sequences or encoding for conserved amino acid residues are expected to alter the biological activity of the encoded peptide. Conserved residues are identical or highly similar sequences in nucleic acids or peptides across homologues with a similar function. Conservation indicates that a sequence has been maintained by natural selection and therefore is critical to its biological function and any changes are expected to alter the biological activity of the encoded peptide.

The amino acid sequences and hexatoxin peptides of the present invention are part of the group of hexatoxins, specifically κ-, ω- and κ/ω-hexatoxins and ω-actinopoditoxins, and are phylogenetically closely related, especially the κ-, ω- and κ/ω-hexatoxins (Pineda et al., BMC Genomics, 2014).

The asparagine amino acid (designated as N) is part of the highly conserved NG amino acid residue and therefore any changes to this amino acid N in the amino acid sequences of the present invention are expected to alter the biological activity of the encoded hexatoxin peptides. It was surprisingly found that by changing the amino acid N in the conserved NG amino acid residue of the amino acid sequences of the present invention, thereby creating the hexatoxin peptides according to the present invention, that the biological activity of the encoded hexatoxin peptides remains similar, while the stability of the hexatoxin peptides in a medium of an alkaline pH-value is also increased.

Table 1 shows amino acid sequences according to the present invention (SEQ ID NOs: 1 to 32 and 34) highlighting the highly conserved NG amino acid residues.

TABLE 1
SEQ ID NOs: 1 to 32 and 34 highlighting the highly
conserved NG amino acid NG residues

| SEQ ID | | |
|---|---|---|
| NO | Peptide name | Amino acid sequence |
| 1 | ω-hexatoxin-Hv1a | SPTCIPSGQPCPYNENCCSQSCTFKENENGNTVKRCD |
| 2 | ω-hexatoxin-Ar1b | GACTPTGQPCPYNESCCSGSCQEQLNENGHTVKRCV |
| 3 | ω-hexatoxin-Hmo1a | APVCTRTDQPCPYNEDCCSGSCTLKKNENGNLVKRCD |
| 4 | ω-hexatoxin-Hmo1c | SPVCTRTDQPCPYDQDCCSGSCTLKKNENGNLVKRCD |
| 5 | ω-hexatoxin-Hmo1b | APVCTRTDQPCPYDQDCCSGSCTLKKNENGNLVKRCD |
| 6 | ω-hexatoxin-Hi1a | STCTPTDQPCPYHESCCSGSCTYKANENGNQVKRCD |
| 7 | ω-hexatoxin-Hi1g | STCTPTDQPCPYDESCCSGSCTYKANENGNQVKRCD |
| 8 | ω-hexatoxin-Hi1c | SSTCIRTDQPCPYNESCCSGSCTYKANENGNQVKRCD |
| 9 | ω-hexatoxin-Hmo1e | STVCIPSGQPCPYSKYCCSGSCTYKENENGNTVQRCD |
| 10 | ω-hexatoxin-Hv1f | SAVCIPSGQPCPYSKYCCSGSCTYKTNENGNSVQRCD |
| 11 | ω-hexatoxin-Hi1d | SPTCIPTGQPCPYNENCCNQSCTYKANENGNQVKRCD |
| 12 | ω-hexatoxin-Hi1b | SPTCIPTGQPCPYNENCCSQSCTYKANENGNQVKRCD |
| 13 | ω-hexatoxin-Hi1f | SPTCIPTGQPCPYNENCCSQSCTYKTNENGNQVKGCD |
| 14 | ω-hexatoxin-Hi1e | SPTCIPTGQPCPYNENCCSQSCTYKANENGNQVKGCD |
| 15 | ω-hexatoxin-Hmo1d | SPVCIPSGQPCPYNEHCCSGSCTYKENENGNTVQRCD |
| 16 | ω-hexatoxin-Ar1a | SSVCIPSGQPCPYNEHCCSGSCTYKENENGNTVQRCD |
| 17 | ω-hexatoxin-Ar1e | SPTCIPSGQPCPYNENCCSKSCTYKENENGNTVQRCD |
| 18 | ω-hexatoxin-Hvn1b | SPTCIPSGQPCPYNENCCSKSCTYKENENGNTVQRCD |
| 19 | ω-hexatoxin-Hv1d | SPTCIPSGQPCPYNENCCSKSCTYKENENGNTVQRCD |
| 20 | ω-hexatoxin-Hf1a | SPTCIRSGQPCPYNENCCSQSCTFKTNENGNTVKRCD |
| 21 | ω-hexatoxin-Hv1b | SSTCIPSGQPCPYNENCCSQSCTYKENENGNTVKRCD |
| 22 | ω-hexatoxin-Hv1c | SSTCIPSGQPCPYNENCCSQSCTFKENENGNTVKRCD |
| 23 | ω-hexatoxin-Ar1d | SPTCIPSGQPCPYNENCCSQSCTFKENENGNTVKRCD |
| 24 | ω-hexatoxin-Hv1e | SPTCIPSGQPCPYNENCCSQSCTYKENENGNTVKRCD |
| 25 | ω/κ-hexatoxin-Hv1h | QYCVPVDQPCSLNTQPCCDDATCTQERNENGHTVYYCRA |
| 26 | ω-actinopoditoxin-Mb1a | SPVCTPSGQPCQPNTQPCCNNAEEEQTINCNGNTVYRCA |
| 27 | κ-hexatoxin-Hv1e | AICPGADRPCAACCPCCPGTSCKAESNGVFYCRKDEP |
| 28 | κ-hexatoxin-Hv1d | VICTGADSPCAACCPCCPGTSCKAESNGVSYCRKDEP |
| 29 | κ-hexatoxin-Hv1c | AICTGADRPCAACCPCCPGTSCKAESNGVSYCRKDEP |
| 30 | κ-hexatoxin-Hv1a | TICTGADRPCAACCPCCPGTSCQGPESNGVVYCRNF |
| 31 | κ-hexatoxin-Hf1a | SPTCTGADRPCAACCPCCPGTSCKGPEPNGVSYCRND |
| 32 | κ-hexatoxin-Hv1b | TICTGADRPCAACCPCCPGTSCQGPEPNGVSYCRND |
| 34 | GS-ω/κ-hexatoxin-Hv1h | GSQYCVPVDQPCSLNTQPCCDDATCTQERNENGHTVYYCRA |

The highly conserved NG amino acid residues are bolded and underlined.

It can be seen that the two peptides can have the same sequences origination from different species—for example, SEQUENCE ID NOs. 1 and 23 or SEQUENCE ID NOs. 17, 18 and 19. The present invention accordingly is based not by the origin of the peptides, but the peptide sequence and the conserved NG amino acid residues.

Methods for introducing nucleotide changes in a sequence encoding a hexatoxin peptide according to the invention, thereby preferably altering the encoded amino acid, are well-known in the art and also referred to as methods for mutagenesis. Examples of methods for mutagenesis are cassette mutagenesis, Kunkel's method, PCR site-directed mutagenesis, site saturation mutagenesis, seamless ligation cloning extract, artificial gene synthesis and CRISPR-Cas9.

Accordingly, the present invention relates to a hexatoxin peptide according to the invention, wherein the amino acid variant on the position is an amino acid A, R, D, C, Q, E, G, H, I, L, K, M, F, P, S, T, W, Y or V.

The present invention also relates to a hexatoxin peptide according to the invention, wherein the amino acid variant on the position is an amino acid A, R, D, C, Q, E, G, H, I, L, K, M, F, P, S, T, W, Y or V; with the proviso that the peptide does not comprise the amino acid sequence of: SEQ ID NO: 33.

The hexatoxin peptide according to the invention may not include aspartic acid. Accordingly, the present invention relates to a hexatoxin peptide according to the invention, wherein the amino acid variant on the position is an amino acid A, R, C, Q, E, G, H, I, L, K, M, F, P, S, T, W, Y or V.

The hexatoxin peptide according to the invention may not include aspartic acid. Accordingly, the present invention relates to a hexatoxin peptide according to the invention, wherein the amino acid variant on the position is an amino acid A, R, C, Q, E, G, H, I, L, K, M, F, P, S, T, W, Y or V; with the proviso that the peptide does not comprise the amino acid sequence of: SEQ ID NO: 33.

The hexatoxin peptide according to the invention may not include aspartic acid, glutamine or glutamic acid. Accordingly, the present invention relates to a hexatoxin peptide according to the invention, wherein the amino acid variant on the position is an amino acid A, R, C, G, H, I, L, K, M, F, P, S, T, W, Y or V.

The hexatoxin peptide according to the invention may not include aspartic acid, glutamine or glutamic acid. Accordingly, the present invention relates to a hexatoxin peptide according to the invention, wherein the amino acid variant on the position is an amino acid A, R, C, G, H, I, L, K, M, F, P, S, T, W, Y or V; with the proviso that the peptide does not comprise the amino acid sequence of: SEQ ID NO: 33.

The hexatoxin peptide according to the invention may not include threonine. Accordingly, the present invention relates to a hexatoxin peptide according to the invention, wherein the amino acid variant on the position is an amino acid A, R, D, C, Q, E, G, H, I, L, K, M, F, P, S, W, Y or V.

The hexatoxin peptide according to the invention may not include threonine or serine. Accordingly, the present invention relates to a hexatoxin peptide according to the invention, wherein the amino acid variant on the position is an amino acid A, R, D, C, Q, E, G, H, I, L, K, M, F, P, W, Y or V.

The hexatoxin peptide according to the invention may not include a charged amino acid. The present invention accordingly relates to a hexatoxin peptide according to the invention, wherein the amino acid variant on the position is an amino acid A, C, Q, G, I, L, M, F, P, S, T, W, Y or V.

The hexatoxin peptide according to the invention may not include a charged amino acid. The present invention accordingly relates to a hexatoxin peptide according to the invention, wherein the amino acid variant on the position is an amino acid A, C, Q, G, I, L, M, F, P, S, T, W, Y or V; with the proviso that the peptide does not comprise the amino acid sequence of: SEQ ID NO: 33.

The hexatoxin peptide according to the invention may not include a charged amino acid or glutamine. The present invention accordingly relates to a hexatoxin peptide according to the invention, wherein the amino acid variant on the position is an amino acid A, C, G, I, L, M, F, P, S, T, W, Y or V.

The hexatoxin peptide according to the invention may not include a charged amino acid or glutamine. The present invention accordingly relates to a hexatoxin peptide according to the invention, wherein the amino acid variant on the position is an amino acid A, C, G, I, L, M, F, P, S, T, W, Y or V; with the proviso that the peptide does not comprise the amino acid sequence of: SEQ ID NO: 33.

The hexatoxin peptide according to the invention may include only nonpolar amino acids. The present invention accordingly relates to a hexatoxin peptide according to the invention, wherein the amino acid variant on the position is an amino acid A, C, G, I, L, M, F, P, W or V.

The present invention also relates to a hexatoxin peptide according to the invention, wherein the amino acid variant on the position is an amino acid A, L, P or T.

The present invention also relates to a hexatoxin peptide according to the invention, wherein the amino acid variant on the position is an amino acid A, L, P or T; with the proviso that the peptide does not comprise the amino acid sequence of: SEQ ID NO: 33. The present invention also relates to a hexatoxin peptide according to the invention, wherein the amino acid variant on the position is an amino acid A, L or P.

The present invention also relates to a hexatoxin peptide according to the invention, wherein the amino acid variant on the position is an amino acid A or L.

Accordingly, in an embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 1, or homologues thereof, wherein the amino acid sequence has at least one amino acid variant on position N29; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 1, wherein the amino acid sequence has at least one amino acid variant on position N29; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 1, or homologues thereof, wherein the amino acid sequence has at least one amino acid variant on position N29; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 29, or homologues thereof, wherein the amino acid sequence has at least one amino acid variant on position N27; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 29, wherein the amino acid sequence has at least one amino acid variant on position N27; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 29, or homologues thereof, wherein the amino acid sequence has at least one amino acid variant on position N27; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 34, or homologues thereof, wherein the amino acid sequence has at least one amino acid variant on position N32; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 34, wherein the amino acid sequence has at least one amino acid variant on position N32; wherein the amino acid variant on the position is an amino acid A or L, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 34, or homologues thereof, wherein the amino acid sequence has at least one amino acid variant on position N32; wherein the amino acid variant on the position is an amino acid A or L, each of which substantially retain the biological activity of the hexatoxin; with the proviso that the peptide does not comprise the amino acid sequence of SEQ ID NO: 33.

The present invention also relates to a hexatoxin peptide according to the invention, wherein the amino acid variant on the position is a non-proteinogenic amino acid.

The variant hexatoxin peptides according to the invention can be produced by chemical synthesis, such as liquid phase peptide synthesis or solid phase peptide synthesis on an automated peptide synthesizer. The variant hexatoxin peptides according to the invention can also be produced by polynucleotides encoding the peptides by recombinant or cell-free expression systems. For forming disulfide bridges between cysteines any method known in the art can be used, for example an oxidation method using a pair of oxidized and reduced glutathiones as described in Klint et al., PLoS One, 2013. Therefore, another aspect of the current invention is to provide a method of preparing a hexatoxin peptide according to the invention.

Advantageously, the variant hexatoxin peptides according to the invention can be produced in yeast expression systems, using for example, Saccharomyces cerevisiae, Pichia pastoris, Hansenula polymorpha, Yarrowia lipolytica, Arxula adeninivorans, Kluyveromyces lactis, and Schizosaccharomyces pombe. It is known in the art, as disclosed in for example WO2013134734A2, that addition of a dipeptide glycine-serine (GS) to the N-terminus of mature pesticidal hexatoxin peptides such as w-hexatoxin-Hv1a, substantially improves the yield of GS-pesticidal hexatoxin peptides when produced by yeast expression systems. The addition of such a glycine-serine dipeptide, does not substantially decrease the biological activity of a pesticidal hexatoxin peptide. The addition of a glycine-serine to the N-terminus of a hexatoxin peptide is preferably done by adding a sequence encoding for a glycine and a serine at the 5′-end of the nucleotide sequence encoding for the hexatoxin peptide.

Accordingly, the present invention relates to optimized variant hexatoxin peptides for improved production in yeast expression systems. The hexatoxin peptide variants of the present invention comprise a N-terminal dipeptide. Preferably, the N-terminal dipeptide has a N-terminal nonpolar amino acid and a C-terminal polar amino acid. More preferably, the N-terminal dipeptide has a N-terminal amino acid selected from glycine, alanine, proline, valine, leucine, isoleucine, phenylalanine and methionine, and a C-terminal amino selected from serine, threonine, cysteine, asparagine, glutamine, histidine, tryptophan and tyrosine. Even more preferably, the N-terminal dipeptide is glycine-serine.

Preferably, a variant of an amino acid sequence is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the non-variant amino acid sequence.

Preferably, a functional variant of an amino acid sequence is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the non-variant amino acid sequence. More preferably, a functional variant is at least 85%, 86%, 87%, 88%, 89%, 90%, 91%, 92%, 93%, 94%, 95%, 96%, 97%, 98% or 99% identical to the non-variant amino acid sequence. Most preferably, a functional variant is at least 90%, 91%, 92%, 93%, 94%, 95%, 96%, 97%, 98% or 99% identical to the non-variant amino acid sequence.

Preferably, a homologue of an amino acid sequence is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any amino acid sequence described herein. More preferably, a homologue of an amino acid sequence is at least 85%, 86%, 87%, 88%, 89%, 90%, 91%, 92%, 93%, 94%, 95%, 96%, 97%, 98% or 99% identical to any amino acid sequence described herein.

A percentage identity between any two nucleic acid sequences, amino acid sequences or peptides can be determined via sequence comparison or sequence alignment. Methods of sequence comparison and sequence alignment are well-known in the art and can be determined via manual alignment and visual inspection or an algorithm, which is suitably implemented on a computer. When performing sequence comparison or alignment one sequence is typically used as a reference sequence to which the other sequence is compared. The comparison occurs in a comparison window which is an individually specified contiguous sequence of each of the compared sequences. Additions or deletions relative from one sequence to the other may be included in any of the sequences, thereby introducing so-called gaps in the other sequence. The introduction of gaps can result in a better alignment between the two sequences. However, the amount of gaps in an alignment should be kept to a minimum in order to create a useful alignment, because too many gaps can cause an alignment to become meaningless. To avoid a high sequence identity between two sequences because of the introduction of too many gaps it is known to a person skilled in the art to use a gap penalty in order to compensate. Gap penalties are used to adjust alignment scores based on the number and length of gaps. Examples of gap penalties are constant, linear, affine, convex, and profile-based gap penalties.

Sequence identity or identity with regard to nucleic acid or amino acid sequences or peptides are understood to mean that the two nucleic acid or amino acid sequences or peptides are the same when aligned for maximum correspondence over a comparison window, as measured using sequence comparison or sequence alignment. Identical, percent identical or percent identity in the context of two or more nucleic acid or amino acid sequences or peptides, refer to two or more nucleic acid or amino acid sequences or peptides that are the same or have a specified percentage of amino acid residues or nucleotides that are the same, when compared and aligned for maximum correspondence over a comparison window, as measured using sequence comparison or sequence alignment. A percentage identity with regard to amino acid sequences or peptides where aligned amino acid positions are not identical is often based on conservative alterations, in particular substitutions, as described above, and is expected to produce a functionally equivalent peptide. The percentage identity can be adjusted upwards to correct for conservative alterations and methods for such adjusting are well-known in the art. For clarity, the percentage identity of a sequence; the “target sequence”, for example a sequence listed herein as a SEQ ID NO, is compared and aligned for maximum correspondence over a comparison window, as measured using sequence comparison or sequence alignment, wherein the length of the individually specified contiguous sequence of the comparison window of the target sequence, for example a sequence listed herein as a SEQ ID NO, is the same or substantially the same as the total length of the target sequence.

Another aspect of the invention relates to the hexatoxin peptide having pesticidal activity when ingested by a pest. The hexatoxin peptide has insecticidal activity when ingested by an insect. To ingest is herein understood to mean to take into the body via the mouth. Pests such as insects possess a complete digestive system consisting of the alimentary canal, an enclosed tube which runs lengthwise through the body from mouth to anus. Food enters the mouth and is processed as it progresses to the anus.

Accordingly, another aspect of the invention relates to a plant, plant tissue or plant propagation material comprising a composition according to the present invention, or having a composition according to the present invention adhered thereto. Preferably, the invention relates to a plant, plant tissue or plant propagation material comprising a composition according to the present invention. Preferably, the invention relates to a plant, plant tissue or plant propagation material having a composition according to the present invention adhered thereto.

Another aspect of the invention relates to a plant, plant tissue or plant propagation material comprising a composition according to the present invention, or having a composition according to the present invention applied thereto. Preferably, the invention relates to a plant, plant tissue or plant propagation material comprising a composition according to the present invention. Preferably, the invention relates to a plant, plant tissue or plant propagation material having a composition according to the present invention applied thereto.

Another aspect of the invention relates to the peptide inhibiting insect voltage-gated ion channel activity or modulating ligand-gated ion channels. The peptides to be substituted on position N27, N28, N29, N30, N31, or N32 according to the invention are described in Table 1 and many of them are known in the art to inhibit insect voltage-gated ion channel activity or to modulate ligand-gated ion channels (Chambers et al., 2019). The amino acid variants on position N27, N28, N29, N30 N31, or N32 according to the invention are expected to not impair the insecticidal activity of the hexatoxin peptides and therefore the peptides are expected to retain their biological activity of inhibiting insect voltage-gated ion channel activity or modulating ligand-gated ion channels.

It is common practice in the art to present pesticidal or insecticidal compounds on or near the plant, plant tissue or plant propagation material in order to have the most effective distribution of the pesticidal or insecticidal compounds towards the pest or the insect. Hereby, pesticidal or insecticidal compounds are delivered to the insect so that they can exert their pesticidal or insecticidal effects.

Accordingly, another aspect of the invention relates to a plant, plant tissue or plant propagation material comprising a hexatoxin peptide according to the present invention, or having a hexatoxin peptide according to the present invention adhered thereto. Preferably, the invention relates to a plant, plant tissue or plant propagation material comprising a hexatoxin peptide according to the present invention. Preferably, the invention relates to a plant, plant tissue or plant propagation material having a hexatoxin peptide according to the present invention adhered thereto.

Plant is herein understood to mean all physical parts of a plant, including seeds, seedlings, saplings, roots, tubers, stems, stalks, foliage and fruits.

Plant tissue is herein understood to mean any type of tissue of a plant, such as epidermis, vascular tissue, ground tissue, xylem, phloem, parenchyma, collenchyma or sclerenchyma.

Plant propagation material is herein understood to mean plant material from which a new plant can be grown, in particular seeds. This also includes plant material suitable for vegetative reproduction and plant cuttings, roots, fruits, tubers, bulbs, corms and rhizomes.

A plant, plant tissue, plant cell or plant propagation material comprising a hexatoxin peptide according to the present invention comprises a plant, plant tissue, plant cell or plant propagation material expressing at least one hexatoxin peptide of the composition.

Another aspect of the invention is a method for delivering a hexatoxin peptide to a pest, the method comprising applying the hexatoxin peptide according to the invention to the plant, plant locus, plant tissue or plant propagation material, to obtain a treated material, wherein the toxin peptide has pesticidal activity when ingested by the pest.

Plant locus is herein understood to mean the locus or the location where the plant or plant propagation material is placed or to be placed. The plant locus includes soil and other vegetation.

Specific methods for applying or administering a peptide according to the invention are known in the art. Examples of these are spraying, dusting, foliar spraying, brushing, spreading, rolling, applying to or coating seeds.

The propagation material can be treated with the hexatoxin peptide during or prior to use such as sowing. The hexatoxin peptide can also be applied to seed kernels, either by soaking the kernels in a liquid composition comprising the hexatoxin peptide or by coating them with a solid composition comprising the hexatoxin peptide.

The hexatoxin peptides according to the present invention are usually applied in compositions further comprising other compounds. The peptides according to the invention can further comprise agriculturally acceptable solid or liquid auxiliaries known in the art such as diluents, carriers, surfactants, stabilizers, antifoams, preservatives, binders, solvents, dispersants or fertilizers. They can also comprise other herbicides or other pesticides such as chemical insecticides, fungicides or nematicides.

The peptides can also be microencapsulated, and such compositions can be prepared using suitable techniques known in the art. Therefore, another aspect of the invention is a composition comprising a pesticidally effective amount of a peptide according to the invention and a suitable carrier or diluent therefor. Pesticidally effective amount is understood to mean an amount that is sufficient to cause a pesticidal effect on the target pest.

Examples of application formulations of compositions according to the invention that can be used for the method according to the invention are solutions, granules, dusts, sprayable powders, emulsion concentrates, coated granules and suspension concentrates.

The formulations can be prepared as pest-ingestible formulations, thereby being suitable to be ingested by a pest, preferably an insect. The suitability of ingestible formulations can be enhanced by adding ethanol or mono- or disaccharide compounds such as glucose, sucrose, fructose or lactose.

Typical rates of concentration of hexatoxin peptides are between 100 and 100000 ppm, preferably between 100 and 10000 ppm or between 1000 and 10000 ppm. Active ingredient is understood to mean the hexatoxin peptide according to the invention. The hexatoxin peptides are generally applied at a rate of from 180 g to 12 kg active ingredient (a.i.) per hectare (ha) depending on the crop and prevailing conditions, such as pest pressure; preferably 180 g to 8 kg ai/ha; more preferably 500 g to 5 kg ai/ha. Rates for soil application (or drenching) and seed treatment application will differ and skilled person would be able to determine the suitable rates based on the conditions, soil, crop, etc.

As described before the purpose of a pesticide is to provide a compound that reduces damage caused by pests, while an insecticide is a compound that reduces damage caused by insects. Accordingly, an aspect of the current invention is to provide a method for controlling pests, comprising applying a peptide according to the invention to a pest or its environment, a plant, plant tissue or plant propagation material or the locus where the plant or plant propagation material is planted. The pest is preferably an insect.

To control is herein understood to mean to kill, to destroy, to retard (or deter or repel) or prevent growth, movement or feeding, to reduce physical fitness or to inhibit reproduction. In addition, an aspect of the current invention is to provide a use of a peptide according to the invention for controlling pests, preferably insects.

The hexatoxin peptides and compositions described herein can be used in a method for controlling pests, preferably insects, with the exception of a method for treatment of the human or animal body by surgery or therapy and diagnostic methods practiced on the human or animal body.

The hexatoxin peptides and compositions according to the invention are preventively and/or curatively valuable active ingredients in the field of pest control, even at low rates of application, which have a very favourable biocidal spectrum and are well tolerated by warm-blooded species, fish and plants. The hexatoxin peptides according to the invention act against all or individual developmental stages of normally sensitive, but also resistant, animal pests, such as insects or representatives of the order Acarina. The insecticidal or acaricidal activity of the hexatoxin peptides according to the invention can manifest itself directly, i.e. in destruction of the pests, which takes place either immediately or only after some time has elapsed, for example during ecdysis, or indirectly, for example in a reduced oviposition and/or hatching rate.

Examples of the above mentioned pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., cephus spp., diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

Pesticides are herein understood to mean substances that are able to control pest infestation, or occurrence of pests. The composition according to the invention can thus be considered to function as a pesticide. Pesticides comprise insecticides, herbicides, nematicides, molluscicides, fungicides and bactericides. Pesticides can for example be in the form of peptides, proteins, microbial agents or chemical pesticides.

Pests are herein understood to mean organisms that cause harm to other organisms, in particular to agricultural crops. To cause harm comprises to kill, to destroy (parts of), to feed upon, to retard or prevent growth, to reduce physical fitness and to inhibit reproduction.

The hexatoxin peptides according to the invention can be used in combination with other compounds, including other pesticides such as insecticides, fungicides, or agents that enhance the activity of the composition according to the invention, in for example chemical treatment or pest control programs. The combination may have further surprising advantages, which could be described as synergistic effects.

Suitable other compounds are, for example, compounds of the following classes of active ingredients: organophosphates, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, benzoylureas, neonicotinoids and biological agents such as Bacillus thuringiensis strains or bacterially-derived pesticides such as spinosads, avermectins and Cry proteins.

The hexatoxin peptides according to the invention can be used in any ratio in combination with other compounds, including other pesticides, insecticides, fungicides or agents that enhance the activity of the hexatoxin peptides according to the invention. Certain ratios are more preferred. A ratio of the hexatoxin peptides according to the invention to other compounds of from 50:1 to 1:50 is herein understood to mean that the total amount of molecules of the hexatoxin peptides according to the invention is from 50 fold to 1/50^{th} of the other compound. Accordingly, the combination has a ratio of the hexatoxin peptides according to the invention to other compounds of from 100:1 to 1:100, from 100:1 to 1:50, from 100:1 to 1:10, from 100:1 to 1:1, from 50:1 to 1:100, from 50:1 to 1:50, from 50:1 to 1:10, from 50:1 to 1:1, from 100:1 to 10:1, from 50:1 to 10:1, from 10:1 to 1:100, from 10:1 to 1:50, from 10:1 to 1:10, from 10:1 to 1:1, from 5:1 to 1:1, from 5:1 to 1:5, from 1:1 to 1:100, from 1:1 to 1:50, from 1:1 to 1:10, from 1:1 to 1:5, from 1:10 to 1:50, from 1:10 to 1:100 or from 1:50 to 1:100. Preferably, the ratio is from 10:1 to 1:100, from 10:1 to 1:50, from 10:1 to 1:10, from 1:1 to 1:100, from 1:1 to 1:50, from 5:1 to 1:5 or from 1:1 to 1:10.

The concentrations of the different active ingredients of the combination of the hexatoxin peptides according to the invention in combination with other compounds can vary depending on the specific formulation, environmental conditions, methods of application and the extent of pesticidal activity of the individual components.

The hexatoxin peptides according to the invention may be the sole active ingredient of a composition or it may be admixed with one or more additional active ingredients such as an insecticide, fungicide, synergist, nutrient, herbicide or plant growth regulator where appropriate.

The hexatoxin peptides according to the invention are normally used in the form of compositions and can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The hexatoxin peptides, and compositions comprising the hexatoxin peptides may be used alone or, preferably, together with the adjuvants conventionally employed in the art of formulation. To this end, it may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects. Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TC, a dispersible concentrate (DC), a wettable powder (WP) or any technically feasible formulation in combination with agriculturally acceptable adjuvants.

Such compositions may be produced in conventional manner, e.g., by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compounds of formula (I) together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

### Plants and Crops:

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. walleriana), iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The toxin peptides according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The toxin peptides are particularly suitable for control of:
a pest of the order Hemiptera, for example, one or more of the species Bemisia tabaci, Aphis craccivora, Myzus persicae, Rhopalosiphum padi, Nilaparvata lugens, and Euschistus heros (preferably in vegetables, soybeans, and sugarcane);
a pest of the order Lepidoptera, for example, one or more of the species Spodoptera littoralis, Spodoptera frugiperda, Plutella xylostella, Cnaphalocrocis medinalis, Cydia pomonella, Chrysodeixis includes, Chilo suppressalis, Elasmopalpus lignosellus, Pseudoplusia includens, and Tuta absoluta (preferably in vegetables and corn);
a pest of the order Thysanoptera, such as the family Thripidae, for example, one or more of Thrips tabaci and Frankliniella occidentalis (preferably in vegetables); and
soil pests (such as of the order Coleoptera), for example, the species Diabrotica balteata, Agriotes spp. and Leptinotarsa decemlineata (preferably in vegetables and corn).

The toxin peptides of the invention are especially suitable for control of one or more of pests selected from mites, thrips, whiteflies, aphids, psyllids, fruit flies, loopers, bollworms, and budworms; in particular for fruit and vegetable crops, whether in greenhouse or field.

The term “crops” is to be understood as including also crop plants that have been so transformed by the use of recombinant DNA techniques that they are capable of synthesising one or more selectively acting toxins. Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

The processes for the preparation of transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l′Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S. A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S. A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603 x MON 810 Maize from Monsanto Europe S. A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

The term “crops” herein is to be understood as to also include crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention may also provide a composition of the invention, for use in controlling parasites in or on an animal. The present invention further provides a composition of the first aspect, for use in controlling ectoparasites on an animal. The present invention further provides a composition of the first aspect, for use in preventing and/or treating diseases transmitted by ectoparasites. The present invention may also provide the use of a composition of the invention, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomma; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephalides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola ovis and Bovicola bovis.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris | Apple, Apricot, Beech, Boxelder, |
| | femorata | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, |
| | | Poplar, Quince, Redbud, |
| | | Serviceberry, Sycamore, Walnut, |
| | | Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, |
| | | Black oak, Cherrybark oak, Water |
| | | oak, Sycamore |
| | Goes tigrinus | Oak |
| | Neoclytus | Ash, Hickory, Oak, Walnut, Birch, |
| | acuminatus | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes | Fig, Alder, Mulberry, Willow, |
| | trilineatus | Netleaf hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, |
| | | Elm, Sourwood, Basswood, |
| | | Honeylocust, Dogwood, |
| | | Eucalyptus, Oak, Hackberry, |
| | | Maple, Fruit trees |
| | Saperda calcarata | Poplar |
| | Strophiona nitens | Chestnut, Oak, Hickory, Walnut, |
| | | Beech, Maple |
| Scolytidae | Corthylus | Maple, Oak, Yellow-poplar, Beech, |
| | columbianus | Boxelder, Sycamore, Birch, |
| | | Basswood, Chestnut, Elm |
| | Dendroctonus | Pine |
| | frontalis | |
| | Dryocoetes betulae | Birch, Sweetgum, Wild cherry, |
| | | Beech, Pear |
| | Monarthrum | Oak, Maple, Birch, Chestnut, |
| | fasciatum | Sweetgum, Blackgum, Poplar, |
| | | Hickory, Mimosa, Apple, Peach, |
| | | Pine |
| | Phloeotribus | Peach, Cherry, Plum, Black cherry, |
| | liminaris | Elm, Mulberry, Mountain-ash |
| | Pseudopityophthorus | Oak, American beech, Black |
| | pruinosus | cherry, Chickasaw plum, Chestnut, |
| | | Maple, Hickory, Hornbeam, |
| | | Hophornbeam |
| Sesiidae | Paranthrene | Oak, American chestnut |
| | simulans | |
| | Sannina | Persimmon |
| | uroceriformis | |
| | Synanthedon | Peach, Plum, Nectarine, Cherry, |
| | exitiosa | Apricot, Almond, Black cherry |
| | Synanthedon | Peach, Plum, Cherry, Beach, Black |
| | pictipes | Cherry |
| | Synanthedon | Tupelo |
| | rubrofascia | |
| | Synanthedon | Dogwood, Pecan, Hickory, Oak, |
| | scitula | Chestnut, Beech, Birch, Black |
| | | cherry, Elm, Mountain-ash, |
| | | Viburnum, Willow, Apple, |
| | | Loquat, Ninebark, Bayberry |
| | Vitacea polistiformis | Grape |

The present invention may be also used to control any pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control pests at various stages of their life cycle, including eggs, larvae, nymphs and adults.

In particular, the present invention may be used to control pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass Ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are: Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The hexatoxin peptides and the compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, urocerus gigas taignus and urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The hexatoxin peptides of the invention are especially suitable for controlling one or more pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae.

The hexatoxin peptide compositions according to the invention are especially suitable for controlling one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp.

The hexatoxin peptide compositions according to the invention are especially suitable for controlling one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padi, and Chilo suppressalis.

In an embodiment, of each aspect, a hexatoxin peptide according to the invention is suitable for controlling Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis in cotton, vegetable, maize, cereal, rice and soya crops.

In an embodiment, a hexatoxin peptide according to the invention is suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The following biological examples serve to illustrate the invention and are non-limitative.

The present invention provides the specific peptides comprising the amino acid sequences of SEQ ID NOs: 35 to 38 as listed in Table 2.

TABLE 2
SEQ ID NOs 35 to 38

| SEQ ID | | |
|---|---|---|
| NO: | Peptide name | Amino acid sequence |
| 35 | Mutant 1 - derived from | SPTCIPSGQPCPYNENCCSQSCTFKENEAGNTVKRCD |
| | SEQ ID NO: 1 | |
| 36 | Mutant 2 - derived from | SPTCIPSGQPCPYNENCCSQSCTFKENELGNTVKRCD |
| | SEQ ID NO: 1 | |
| 37 | Mutant 3 - derived from | AICTGADRPCAACCPCCPGTSCKAESLGVSYCRKDEP |
| | SEQ ID NO: 29 | |
| 38 | Mutant 4 - derived from | GSQYCVPVDQPCSLNTQPCCDDATCTQERNELGHTVYYCRA |
| | SEQ ID NO: 34 | |

An embodiment of the invention is a hexatoxin peptide comprising an amino acid sequence of any one of SEQ ID NOs: 35 to 38. Another embodiment of the invention is a hexatoxin peptide comprising an amino acid sequence of any one of SEQ ID NOs: 35 or 36. Yet another embodiment of the invention is a hexatoxin peptide comprising an amino acid sequence of any one of SEQ ID NOs: 37 or 38. Another embodiment of the invention is a hexatoxin peptide comprising an amino acid sequence of SEQ ID NO 35. Another embodiment of the invention is a hexatoxin peptide comprising an amino acid sequence of SEQ ID NO 36. Another embodiment of the invention is a hexatoxin peptide comprising an amino acid sequence of SEQ ID NO 38. Another embodiment of the invention is a hexatoxin peptide comprising an amino acid sequence of SEQ ID NO 38. In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 35, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 35, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 35, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 36, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 36, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 36, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 37, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 37, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 37, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO: 38, or homologues thereof; each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is at least 65%, 70%, 75%, 80%, 85%, 90%, 91%, 92%, 93%, 94%, 95%, 96%, 97%, 98% or 99% identical to SEQ ID NO 38, each of which substantially retain the biological activity of the hexatoxin; with the proviso that the peptide does not comprise the amino acid sequence of SEQ ID NO: 33.

In another embodiment the present invention relates to a hexatoxin peptide variant comprising an amino acid sequence which is SEQ ID NO: 38, or homologues thereof; each of which substantially retain the biological activity of the hexatoxin; with the proviso that the peptide does not comprise the amino acid sequence of SEQ ID NO: 33.

### EXAMPLES

The following biological examples serve to illustrate the invention and are non-limitative.

### Example 1: Insecticidal Effect of ω-Hexatoxin-Hv1a, Mutant 1 and Mutant 2 Against Spodoptera littoralis L1

Cotton leaf discs were placed onto agar in 24-well microtiter plates and sprayed with aqueous test solutions prepared from 10,000 ppm DMSO stock solutions. After drying the leaf discs were infested with five L1 larvae. The samples were assessed for mortality in comparison to untreated samples 6 days after infestation. Results of observed mortality are shown in Table C below.

TABLE C
observed mortality of Spodoptera littoralis L1

| | 1250 | 2500 | 5000 | 10000 |
|---|---|---|---|---|
| Test solution | ppm | ppm | ppm | ppm |
| ω-hexatoxin-Hv1a | 13% | 0% | 7% | 27% |
| (SEQ ID NO: 1) | | | | |
| Mutant 1 (SEQ ID NO: 35) | 7% | 7% | 20% | 7% |
| Mutant 2 (SEQ ID NO: 36) | 13% | 7% | 13% | 20% |

### Example 2: Insecticidal Effect of ω-Hexatoxin-Hv1 a, Mutant 1 and Mutant 2 Against Plutella xylostella L2

24-well plates with artificial diet were treated with aqueous test solutions prepared from 10,000 ppm DMSO stock solutions by pipetting. After drying, the plates were infested with L2 larvae (10 to 15 per well). The samples were assessed for mortality in comparison to untreated samples 5 days after infestation. Results of observed mortality are shown in Table D below.

TABLE D
observed mortality of Plutella xylostella L2

| | 1250 | 2500 | 5000 | 10000 |
|---|---|---|---|---|
| Test solution | ppm | ppm | ppm | ppm |
| ω-hexatoxin-Hv1a | 33% | 75% | 88% | 100% |
| (SEQ ID NO: 1) | | | | |
| Mutant 1 (SEQ ID NO: 35) | 25% | 46% | 79% | 100% |
| Mutant 2 (SEQ ID NO: 36) | 42% | 63% | 92% | 100% |

### Example 3: Stability Assays

ω-hexatoxin-Hv1 a, mutant 1 and mutant 2 peptides were subjected to a stability assay. Each peptide was incubated separately in an aqueous buffer at 25° C. adjusted to pH 8 for 24, 48, 72, 96 or 168 h. The degree of deamidation was assessed by mass spectrometry as a percentage of predicted total possible deamidation. Results of deamidation are shown in Table F below

TABLE E
observed deamidation

| Peptide | 0 h | 24 h | 48 h | 72 h | 96 h | 168 h |
|---|---|---|---|---|---|---|
| ω-hexatoxin-Hv1a | 0% | 24% | 48% | 66% | 77% | 90% |
| (SEQ ID NO: 1) | | | | | | |
| Mutant 1 (SEQ ID NO: | 0% | n/a | 7% | 9% | 11% | 15% |
| 35) | | | | | | |
| Mutant 2 (SEQ ID NO: | 0% | n/a | 7% | 9% | 11% | 15% |
| 36) | | | | | | |

“n/a” indicates no data

### Example 4: Stability Assays

ω-hexatoxin-Hv1a, K-hexatoxin-Hv1c, GS-w/K-hexatoxin-Hv1h, mutant 2, mutant 3 and mutant 4 peptides were subjected to a stability assay. Each peptide was incubated separately in an aqueous buffer at 25° C. adjusted to pH 9 for 24, 48, 72, 96, 120, 144, 168, 240, 360, 480 h. The degree of deamidation was assessed by LCMS as a percentage of predicted total possible deamidation. Results of deamidation are shown in Table G below.

TABLE F
observed deamidation

| Peptide | 0 h | 24 h | 48 h | 72 h | 96 h | 120 h | 144 h | 168 h | 240 h | 360 h | 480 h |
|---|---|---|---|---|---|---|---|---|---|---|---|
| ω-hexatoxin- | 0% | 32% | 48% | 68% | 85% | 96% | 100% | 100% | n/a | n/a | n/a |
| Hv1a (SEQ | | | | | | | | | | | |
| ID NO: 1) | | | | | | | | | | | |
| κ-hexatoxin- | 0% | 8% | 19% | 24% | 38% | 47% | 57% | 54% | n/a | n/a | n/a |
| Hv1c (SEQ | | | | | | | | | | | |
| ID NO: 29) | | | | | | | | | | | |
| GS-ω/κ- | 0% | 5% | 8% | 9% | 14% | 15% | 18% | 19% | n/a | n/a | n/a |
| hexatoxin- | | | | | | | | | | | |
| Hv1h (SEQ | | | | | | | | | | | |
| ID NO. 34) | | | | | | | | | | | |
| Mutant 2 | 0% | n/a | n/a | n/a | n/a | 5% | n/a | n/a | 15% | 19% | 25% |
| (SEQ ID NO: | | | | | | | | | | | |
| 36) | | | | | | | | | | | |
| Mutant 3 | 0% | n/a | n/a | n/a | n/a | 0% | n/a | n/a | 0% | 0% | 0% |
| (SEQ ID NO: | | | | | | | | | | | |
| 37) | | | | | | | | | | | |
| Mutant 4 | 0% | n/a | n/a | n/a | n/a | 0% | n/a | n/a | 4% | 9% | 16% |
| (SEQ ID NO: | | | | | | | | | | | |
| 38) | | | | | | | | | | | |

“n/a” indicates no data

### Example 5: Stability Assays

ω-hexatoxin-Hv1a, κ-hexatoxin-Hv1c, GS-ω/κ-hexatoxin-Hv1h, mutant 2, mutant 3 and mutant 4 peptides were subjected to a stability assay. Each peptide was incubated separately in an aqueous buffer at 25° C. adjusted to pH 9 for 24, 48, 72, 96, 120, 144, 168, 240, 360, 480 h. The degree of stability was assessed by LCMS as the percentage of remaining parent peak. Results of the degree of observed stability are shown in Table H below

TABLE G
observed overall stability

| Peptide | 0 h | 24 h | 48 h | 72 h | 96 h | 120 h | 144 h | 168 h | 240 h | 360 h | 480 h |
|---|---|---|---|---|---|---|---|---|---|---|---|
| ω- | 100% | 65% | 48% | 28% | 11% | 3% | 0% | 0% | n/a | 0 | n/a |
| hexatoxin- | | | | | | | | | | | |
| Hv1a (SEQ | | | | | | | | | | | |
| ID NO: 1) | | | | | | | | | | | |
| κ-hexatoxin- | 100% | 91% | 80% | 75% | 61% | 51% | 41% | 30% | n/a | 9% | n/a |
| Hv1c (SEQ | | | | | | | | | | | |
| ID NO: 29) | | | | | | | | | | | |
| GS-ω/κ- | 100% | 94% | 91% | 82% | 83% | 83% | 81% | 69% | n/a | 50% | n/a |
| hexatoxin- | | | | | | | | | | | |
| Hv1h (SEQ | | | | | | | | | | | |
| ID NO. 34) | | | | | | | | | | | |
| Mutant 2 | 100% | n/a | n/a | n/a | n/a | 94% | n/a | n/a | 79% | 73% | 65% |
| (SEQ ID | | | | | | | | | | | |
| NO: 36) | | | | | | | | | | | |
| Mutant 3 | 100% | n/a | n/a | n/a | n/a | 100% | n/a | n/a | 100% | 100% | 100% |
| (SEQ ID | | | | | | | | | | | |
| NO: 37) | | | | | | | | | | | |
| Mutant 4 | 100% | n/a | n/a | n/a | n/a | 95% | n/a | n/a | 89% | 90% | 82% |
| (SEQ ID | | | | | | | | | | | |
| NO: 38) | | | | | | | | | | | |

“n/a” indicates no data

The above examples illustrate the enhanced stability and efficacy of the peptides according to the invention. Accordingly, the present invention also advantageously also relates to the peptides set out in the examples, their use in compositions for application, and to plant material or plant locus treated with, or comprising the peptides and/or compositions.

### REFERENCES

Chambers C., Cutler P., Huang Y., Goodchild J. A., Blythe J., Wang C. K., Bigot A., Kaas Q., Craik D. J., Sabbadin D., Earley F. G.; Insecticidal spider toxins are high affinity positive allosteric modulators of the nicotinic acetylcholine receptor; FEBS Letters, 593(12), 1336-1350, 2019. https://doi.org/10.1002/1873-3468.13435
Diepold K., Bomans K., Wiedmann M., Zimmermann, B., Petzold A., Schlothauer T., Mueller R., Moritz B., Stracke J. O., Molhoj M., Reusch D., Bulau P.; Simultaneous Assessment of Asp Isomerization and Asn Deamidation in Recombinant Antibodies by LC-MS following Incubation at Elevated Temperatures; PLoS ONE, 2012. https://doi.org/10.1371/journal.pone.0030295
Dow J. A.; pH Gradients In Lepidopteran Midgut; Journal of Experimental Biology, 172(1), 355-375, 1992.
Gfeller D., Michielin O., Zoete V.; SwissSidechain: a molecular and structural database of non-natural sidechains; Nucleic Acids Research, 41, D1, D327-D332, 2013. https://doi.org/10.1093/nar/gks991
Khan S. A., Zafar Y., Briddon R. W., Malik, K. A., Mukhtar Z.; Spider venom toxin protects plants from insect attack; Transgenic Research, 15, 349-357, 2006. https://doi.org/10.1007/s11248-006-0007-2
Klint J. K., Senff S., Saez N. J., Seshadri R., Lau H. Y., Bende N. S., Undheim E. A. B., Rash L. D., Mobli M., King G. F.; Production of Recombinant Disulfide-Rich Venom Peptides for Structural and Functional Analysis via Expression in the Periplasm of E. coli; PloS ONE, 2013. https://doi.org/10.1371/journal.pone.0063865
Pineda S. S., Sollod B. L., Wilson D., Darling A., Sunagar K., Undheim E. A. B., Kely, L., Antunes A., Fry B. G., King G. F.; Diversification of a single ancestral gene into a successful toxin superfamily in highly venomous Australian funnel-web spiders; BMC Genomics, 15: 177, 2014. https://dx.doi.org/10.1186%2F1471-2164-15-177
Pineda S. S., Chaumeil P., Kunert A., Kaas Q., Thang M. W. C., Le L., Nuhn M., Herzig V., Saez N.J., Cristofori-Armstrong B., Anangi R., Senff S., Gorse D., King G. F.; ArachnoServer 3.0: an online resource for automated discovery, analysis and annotation of spider toxins; Bioinformatics, 34(6), 1074-1076, 2018. https://doi.org/10.1093/bioinformatics/btx661
Windley M. J., Herzig V., Dziemborowicz S. A., Hardy M. C., King G. F., Nicholson G. M.; Spider-Venom Peptides as Bioinsecticides; Toxins, 4(3), 191-227, 2012. https://doi.org/10.3390/toxins4030191

1. A hexatoxin peptide variant comprising
a. an amino acid sequence which is at least 90% identical to any one of SEQ ID NOs: 27 to 29, wherein the amino acid sequence has at least one amino acid variant on position N27;
b. an amino acid sequence which is at least 90% identical to any one of SEQ ID NOs: 2, 6, 7, 30 or 32, wherein the amino acid sequence has at least one amino acid variant on position N28;
c. an amino acid sequence which is at least 90% identical to any one of SEQ ID NOs: 1, 3, 4, 5, 8 to 24 or 31, wherein the amino acid sequence has at least one amino acid variant on position N29;
d. an amino acid sequence which is at least 90% identical to SEQ ID NO: 25, wherein the amino acid sequence has at least one amino acid variant on position N30;
e. an amino acid sequence which is at least 90% identical to SEQ ID NOs: 26, wherein the amino acid sequence has at least one amino acid variant on position N31; or
f. an amino acid sequence which is at least 90% identical to SEQ ID NO: 34, wherein the amino acid sequence has at least one amino acid variant on position N32;
each of which retain the biological activity of the hexatoxin; with the proviso that the peptide does not comprise the amino acid sequence of SEQ ID NO: 33.

2. The peptide according to claim 1, wherein the respective amino acid sequences are at least 91%, 92%, 93%, 94%, 95%, 96%, 97%, 98% or 99% identical to an amino acid sequence of any one of the respective SEQ ID NOs according to (a) to (f), with the exception of the amino acid variant at the respective position.

3. The peptide according to claim 1, wherein the respective amino acid sequences are substantially identical to an amino acid sequence of any one of the respective SEQ ID NOs according to (a) to (f), with the exception of the amino acid variant at the respective position.

4. The peptide according to claim 1, comprising
a. an amino acid sequence of any of SEQ ID NOs: 27 to 29, wherein the amino acid sequence has an amino acid variant on position N27; or
b. an amino acid sequence of any one of SEQ ID NOs: 2, 6 or 7, wherein the amino acid sequence has an amino acid variant on position N28; or
c. an amino acid sequence of any one of SEQ ID NOs: 1, 3, 4, 5 or 8 to 24, wherein the amino acid sequence has an amino acid variant on position N29; or
d. an amino acid sequence of SEQ ID NO: 26, wherein the amino acid sequence has an amino acid variant on position N31; or
e. an amino acid sequence of SEQ ID NO: 34, wherein the amino acid sequence has an amino acid variant on position N32.

5. The peptide according to claim 1, comprising an amino acid sequence which is at least 90%, 91%, 92%, 93%, 94%, 95%, 96%, 97%, 98% or 99% identical to an amino acid sequence of SEQ ID NO: 1, wherein the amino acid sequence has an amino acid variant on position N29.

6. The peptide according to claim 1, wherein the peptide comprises an amino acid sequence of SEQ ID NO: 1, wherein the amino acid sequence has an amino acid variant on position N29.

7. The peptide according to claim 1, wherein the peptide comprises an amino acid sequence of SEQ ID NO: 1, wherein the amino acid sequence has an amino acid variant on position N29.

8. The peptide according to claim 1, wherein the amino acid variant on the respective position is an amino acid A, L, P or T.

9. A composition comprising a pesticidally effective amount of a peptide according to claim 1, and a suitable carrier and/or diluent.

10. A plant, plant tissue or plant propagation material comprising the peptide according to claim 1, or having the peptide according to claim 1 applied thereto.

11. A method for increasing stability of a hexatoxin peptide, the method comprising introducing an amino acid variant on position N27 into an amino acid sequence that is at least 90%, 91%, 92%, 93%, 94%, 95%, 96%, 97%, 98% or 99% identical to an amino acid sequence comprising any one of SEQ ID NOs: 27 to 29, on position N28 into an amino acid sequence that is at least 90%, 91%, 92%, 93%, 94%, 95%, 96%, 97%, 98% or 99% identical to an amino acid sequence comprising any one of SEQ ID NOs: 2, 6, 7, 30 or 32, on position N29 into an amino acid sequence that is at least 90%, 91%, 92%, 93%, 94%, 95%, 96%, 97%, 98% or 99% identical to an amino acid sequence comprising any one of SEQ ID NOs: 1, 3, 4, 5, 8 to 24 or 31, on position N30 into an amino acid sequence that is at least 90%, 91%, 92%, 93%, 94%, 95%, 96%, 97%, 98% or 99% identical to an amino acid sequence comprising SEQ ID NO: 25, on position N31 into an amino acid sequence that is at least 90%, 91%, 92%, 93%, 94%, 95%, 96%, 97%, 98% or 99% identical to an amino acid sequence comprising SEQ ID NOs: 26, or on position N32 into an amino acid sequence that is at least 90%, 91%, 92%, 93%, 94%, 95%, 96%, 97%, 98% or 99% identical to an amino acid sequence comprising SEQ ID NO: 34.

12. The method according to claim 11, wherein the increased stability is determined by comparing the stability of the hexatoxin peptide to its non-variant amino acid sequence in an alkaline medium of from pH 7 to pH 14.

13. The method according to claim 12, wherein the increased stability is monitored in an alkaline medium in the range of from pH 7 to pH 14, and at a temperature of from 25° C. to 42° C., over a period of at least two days.

14. A method for delivering a hexatoxin peptide to an insect, the method comprising applying the peptide according to claim 1 to the plant, plant locus, plant tissue or plant propagation material, to obtain a treated material.

15. A method for controlling pest infestations, comprising applying a hexatoxin peptide according to claim 1 to a pest or its environment, a plant, plant tissue or plant propagation material or the locus where the plant or plant propagation material is planted.

16. Use of a hexatoxin peptide according to claim 1 for controlling pest infestations.
